(19) United States
(12) Patent Application Publication
(10) Pub. No.: US 2026/0233032 A1
(43) Pub. Date: Aug. 13, 2026

(54) HISTOTRIPSY SYSTEMS AND METHODS

(71) Applicant: The Regents of the University of Michigan, Ann Arbor, MI (US)

(72) Inventors: Timothy L. HALL, Ann Arbor, MI (US); Zhen XU, Ann Arbor, MI (US); Greyson STOCKER, Ann Arbor, MI (US)

(21) Appl. No.: 19/475,240

(22) PCT Filed: Apr. 15, 2024
(86) PCT No.: PCT/US2024/024674

§ 371 Date: Oct. 14, 2025

Related U.S. Application Data
(60) Provisional application No. 63/496,181, filed on Apr. 14, 2023.

(51) Int. Cl. A61N 7/00

(52) U.S. Cl. CPC A61N 7/00; A61N 2007/0052; A61N 2007/006; A61N 2007/0065; A61N 2007/0078

(57) ABSTRACT

A histotripsy therapy system configured for the treatment of tissue is provided, which may include any number of features. Provided herein are systems and methods that provide efficacious non-invasive and minimally invasive therapeutic, diagnostic and research procedures. The systems provided herein can include flat histotripsy arrays that provide for dry coupling to a patient without a water bath. Dual-material acoustic lenses are provided which facilitate the histotripsy array or probe having a flat coupling surface. Histotripsy transducer arrays are also provided with a conductive material embedded in a matching layer of the transducer array to provide for a high-voltage connection to the transmitting surface of the array.

### PRIORITY CLAIM

This patent application claims priority to U.S. provisional patent application No. 63/496,181, titled “HISTOTRIPSY SYSTEMS AND METHODS”, and filed on Apr. 14, 2023, which is herein incorporated by reference in its entirety.

### STATEMENT AS TO FEDERALLY SPONSORED RESEARCH

This invention was made with Government support under Grant No. R01-CA211217 awarded by the National Institutes of Health. The Government has certain rights in the invention.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure details novel high intensity therapeutic ultrasound (HITU) systems configured to produce acoustic cavitation, methods, devices and procedures for the minimally and non-invasive treatment of healthy, diseased and/or injured tissue. The acoustic cavitation systems and methods described herein, also referred to Histotripsy, may include transducers, drive electronics, positioning robotics, imaging systems, and integrated treatment planning and control software to provide comprehensive treatment and therapy for soft tissues in a patient.

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) Histotripsy produces lesions in a controlled and precise manner. It is important to emphasize that unlike thermal ablative technologies such as microwave, radiofrequency, high-intensity focused ultrasound (HIFU) cryo or radiation, Histotripsy relies on the mechanical action of cavitation for tissue destruction and not on heat, cold or ionizing energy.

Typical histotripsy transducers consist of multiple piezoelectric elements, arranged in a spherical geometry to allow for high focal gain. To further aid in generating the high focal gain, most transducers feature an f #less than 1. Transducers can be constructed from either many flat, individual elements, or a large piezoceramic machined into multiple elements. Both strategies have been used successfully multiple times, however, the latter requires specialized tooling, increasing the effective difficulty of such a method for many scientific labs constructing their own instrumentation. Most commonly, histotripsy transducers are designed with an operating frequency in the range of 500 kHz-1.5 MHz, though transducers have been constructed at frequencies as low as 250 kHz, and as high as 6.8 MHz for specific applications. Elements are bonded to acoustic matching layers or lenses, and are electrically insulated to allow for safe operation at high driving voltages.

The simplest form of histotripsy transducer is the single focus transducer. Most commonly, this type of transducer is designed using multiple flat piezoelectric elements bonded to acoustic lenses. Oftentimes, the acoustic lenses are 3D printed, and can be integrated into a transducer scaffold for the highest degree of simplicity. The transducer is designed such that all lenses are confocal, allowing very high focal gain at the geometric focus of the transducer, even when using a relatively small number of elements. Designs of this nature allow for fabrication to be simple and inexpensive, and also simplify the associated driving electronics, as the requisite quantity of driving channels can be low.

Alternatively to single focus transducers, phased arrays are made of many elements, featuring flat acoustic matching layers (as opposed to acoustic lenses). Elements are typically arranged in a spherical geometry, such that constructive interference at the transducer's focus allows for high focal gain, and the focal pressures necessary to perform histotripsy. Histotripsy phased arrays can feature hundreds of elements, and thus are significantly more complicated and costly to build compared to single focus transducers. Additionally, phased array electrical driving systems allow for individual elements to be driven with arbitrary phase with respect to other elements within the array, enabling advanced treatment techniques such as EFS.

Histotripsy is typically applied transcutaneously, as this is the most non-invasive treatment option, however, there are areas of the body with very limited acoustic access that complicate transcutaneous treatment. These areas (such as within the pelvis and rib cage) could benefit from and endocavity, endoscopic, or laparoscopic form-factor transducer which would bypass the acoustic window limitations by being inserted into either a natural orifice or a small laparoscopic port. This would allow histotripsy treatment of these regions while maintaining a minimally invasive approach. Potential applications of endocavity or laparoscopic histotripsy include treatment of prostate cancer, BPH, uterine fibroids, endometriosis, pelvic abscess, and pancreatic cancer, among others. All of these applications could be treated via insertion into a natural orifice (rectum, vagina, or stomach), allowing for non-invasive treatment to be preserved.

Previous efforts focused on developing endoscopic histotripsy transducers have focused on high frequency devices for precise ablation. Specifically, these devices have been designed with the intent on treating brain tumors via a burr hole in the skull, allowing for highly precise targeting and ablation. Focal gain is proportional to the square of the operating frequency, and thus, increasing frequency to greater than 5 MHz allows for miniaturization into the 5×5 mm size range. Increasing frequency also decreases the focal zone size, making these devices ideally suited for the precision ablations they were designed for, however, they would be unsuitable for large volume ablation applications due to the long treatment times that would be required. Additionally, these devices featured short working distances, which would eliminate the possibility of treating many indications via insertion into a natural orifice.

For regions of the body with a suitable acoustic window, transcutaneous histotripsy treatment is preferred, as it provides the most non-invasive approach. Most notably, transcutaneous histotripsy has been developed and is under clinical investigation for the treatment of liver cancer. Additionally, significant pre-clinical work has been performed, developing transcutaneous histotripsy for the treatment of DVT, STS, renal cancer, brain cancer, and ICH, among others. Histotripsy is ideally suited for non-invasive treatment of large masses, as it does not suffer from the diffusive (heat or chemical) effects of other minimally invasive ablation modalities such as radio-frequency ablation (RFA), microwave ablation, or percutaneous ethanol injection.

Previously developed modular transcutaneous histotripsy instrumentation typically feature piezoelectric elements encased in 3D printed housings. These designs often feature low aperture utilization (50-60% at maximum), which can decrease peak pressure outputs and limit the range of locations within the body where histotripsy can be performed. Additionally, previous work has shown that treatment parameters such as pulse repetition frequency (PRF) can have a dramatic effect on ablation efficiency. As PRF is increased, it becomes more likely that previous cavitation nuclei can be re-excited, generating new cavitation clouds closely corresponding to previously generated clouds, decreasing the actual new damage realized by each pulse. While efficient per-pulse ablations can be performed by simply decreasing PRF, this leads to slow overall treatment rates.

Phased arrays offer several advantages over conventional, single focus histotripsy transducers. One such advantage is the ability to perform EFS, which can be used to enhance ablation efficiency, resulting in higher treatment rates. By specifically designing EFS steering sequences to low local PRFs, while maintaining high global PRFs, allowing for rapid and per-pulse efficient ablations. While ablation rates were increased in these studies dramatically, the results were partially dependent on the low frequency (250 kHz), hemispherical array, which is to suitable for treating many indications. In addition to rapid ablation EFS techniques, phased arrays allow for other advanced treatment capabilities, such as the ability to perform aberration correction (AC) and treatment monitoring techniques.

### SUMMARY OF THE DISCLOSURE

An ultrasound device is provided, comprising a flat transducer array comprising one or more transducer elements, a dual-material acoustic lens coupled to the flat transducer array, the dual-material acoustic lens having a flat coupling surface and comprising a first material having a first speed of sound therein and a second material having a second speed of sound therein, the dual-material acoustic lens being configured to provide an equal time of flight between the flat transducer array and a focus of the ultrasound device for any radial distance across the flat transducer array.

In some aspects, the dual-material acoustic lens has a constant overall thickness.

In other aspects, the first speed of sound in the first material is higher than the second speed of sound in the second material.

In one aspect, the first speed of sound is greater than 1500 m/s.

In other aspects, the second speed of sound is less than 1500 m/s.

In one aspect, the first material has a concave shape.

In other aspects, the first material has an elliptical shape.

In one aspect, the first material comprises 3D printed plastic.

In other aspects, the second material comprises a silicon fill material.

In one aspect, the dual-material acoustic lens comprises a Fresnel lens.

In other aspects, the first material is shaped to include a plurality of steps.

In some aspects, each of the plurality of steps has a thickness that is an integer multiple of a wavelength of the flat transducer array.

In another aspect, the dual-material acoustic lens has a thickness of less than 15 mm.

In some aspects, the dual-material acoustic lens has a thickness of less than 13 mm.

In one aspect, the dual-material acoustic lens has a thickness of less than 5 mm.

In another aspect, the dual-material acoustic lens has a thickness of less than 4 mm.

In one aspect, the dual-material acoustic lens and the flat transducer array are disposed on or in a probe housing.

In other aspects, the dual-material acoustic lens provides for dry coupling of the flat transducer array to a subject.

In one aspect, the device is configured to deliver histotripsy pulses into the subject's tissue to generate cavitation at the focus.

In one aspect, the device further includes an ultrasound imaging transducer array integrated into a central location of the dual-material acoustic lens and/or the flat transducer array.

An ultrasound device is also provided, comprising: a housing; a flat transducer array comprising one or more transducer elements disposed in the housing; electrical cabling disposed at least partially in the housing and configured to provide an electrical connection to a back surface of each of the one or more transducer elements; and a matching layer coupled to a transmitting surface of the flat transducer array, the matching layer having a conductive material embedded in the matching layer, the conductive material being configured to provide an electrical connection to each of the one or more transducer elements at the transmitting surface.

In some aspects, the conductive material comprises a conductive mesh.

In one aspect, the conductive material comprises a conductive grid.

In another aspect, the conductive material comprises a copper mesh.

In some aspects, the matching layer is directly bonded to the transmitting surface of the flat transducer array.

In another aspect, the flat transducer array is directly bonded to the matching layer before being diced into a plurality of transducer elements.

In one aspect, the matching layer comprises a dual-material acoustic lens coupled to the flat transducer array, the dual-material acoustic lens having a flat coupling surface and comprising a first material having a first speed of sound therein and a second material having a second speed of sound therein, the dual-material acoustic lens being configured to provide an equal time of flight between the flat transducer array and a focus of the ultrasound device for any radial distance across the flat transducer array.

In some aspects, the dual-material acoustic lens has a constant overall thickness.

In other aspects, the first speed of sound in the first material is higher than the second speed of sound in the second material.

In one aspect, the first speed of sound is greater than 1500 m/s.

In other aspects, the second speed of sound is less than 1500 m/s.

In one aspect, the first material has a concave shape.

In other aspects, the first material has an elliptical shape.

In one aspect, the first material comprises 3D printed plastic.

In other aspects, the second material comprises a silicon fill material.

In one aspect, the dual-material acoustic lens comprises a Fresnel lens.

In other aspects, the first material is shaped to include a plurality of steps.

In some aspects, each of the plurality of steps has a thickness that is an integer multiple of a wavelength of the flat transducer array.

In another aspect, the dual-material acoustic lens has a thickness of less than 15 mm.

In some aspects, the dual-material acoustic lens has a thickness of less than 13 mm.

In one aspect, the dual-material acoustic lens has a thickness of less than 5 mm.

In another aspect, the dual-material acoustic lens has a thickness of less than 4 mm.

In one aspect, the dual-material acoustic lens and the flat transducer array are disposed on or in a probe housing.

In other aspects, the dual-material acoustic lens provides for dry coupling of the flat transducer array to a subject.

In one aspect, the device is configured to deliver histotripsy pulses into the subject's tissue to generate cavitation at the focus.

In one aspect, the device further includes an ultrasound imaging transducer array integrated into a central location of the dual-material acoustic lens and/or the flat transducer array.

In some aspects, the conductive material is embedded in the first material.

In one aspect, the device further includes a printed circuit board configured to receive a spring pin electrical connector for each of the one or more transducer elements, the spring pin electrical connector providing an electrical connection between the electrical cabling and the back surface of each of the one or more transducer elements.

In another aspect, the device comprises an endocavity histotripsy probe or a laparoscopic histotripsy probe.

A flat histotripsy therapy array is provided, comprising: a plurality of histotripsy transducer elements arranged on a flat surface and configured to enable acoustic coupling with a patient without requiring water coupling.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1B illustrate an ultrasound imaging and therapy system.

FIG. 2 provides a graphical representation of flat front profile determination parameters for a flat profile lens.

FIGS. 3A-3B show lens profiles for elliptical, flat-front dual material, and flat-front Fresnel dual material lenses.

FIGS. 4A-4B show a laparoscopic transducer.

FIGS. 5A-5B show a flat endocavity array design.

FIG. 6 shows a soft tissue sarcoma (STS) transducer array.

### DETAILED DESCRIPTION

The system, methods and devices of the disclosure may be used for non-invasive acoustic cavitation for the treatment of healthy, diseased and/or injured tissue including but not limited to tissue destruction, cutting, skeletonizing and ablation. Furthermore, due to tissue selective properties, histotripsy may be used to create a cytoskeleton that allows for subsequent tissue regeneration either de novo or through the application of stem cells and other adjuvants. Finally, histotripsy can be used to cause the release of delivered agents such as chemotherapy and immunotherapy by locally causing the release of these agents by the application of acoustic energy to the targets. As will be described below, the acoustic cavitation system may include various sub-systems, including a Cart, Therapy, Integrated Imaging, Robotics, Coupling and Software. The system also may comprise various Other Components, Ancillaries and Accessories, including but not limited to computers, cables and connectors, networking devices, power supplies, displays, drawers/storage, doors, wheels, and various simulation and training tools, etc. All systems, methods and means creating/controlling/delivering histotripsy are considered to be a part of this disclosure, including new related inventions disclosed herein.

Histotripsy therapy is typically guided by ultrasound imaging. Histotripsy can also be guided by MRI. This disclosure describes novel systems (hardware and software) and methods for simultaneous ultrasound and MRI guidance and monitoring for histotripsy treatment. This disclosure describes systems and methods that use magnetic resonance (MR) thermometry combined with low temperature focused ultrasound (FUS) heating or MR-acoustic radiation force imaging (MR-ARFI) to accurately predict a locus of histotripsy cavitation. Further, histotripsy-generated ablation (e.g., brain ablation) can be visualized using diffusion-weighted MRI (dMRI).

This disclosure also provides techniques to enhance MR image quality by integrating ultrasound-translucent RF receive coils into the histotripsy system. Additionally, driving electronics are provided that permit a single amplifier setup to both transmit histotripsy acoustic pulses and receive subsequent acoustic cavitation emission (ACE) signals. This disclosure provides ACE-based quantitative cavitation monitoring to enable real-time treatment ultrasound monitoring at a high frame rate (≥50 Hz) that is practical for histotripsy delivery as well as cavitation mapping of on-target and off-target locations including on a bone (e.g., skull) surface. The cavitation mapping can be complemented by periodic updates from dMRI to provide tissue damage evaluation during histotripsy with both high spatial and temporal resolution.

This disclosure also provides color-encoded cavitation/damage maps based on ACE signals that can be co-registered and superimposed over MR images to form integrated US and MR guidance.

This disclosure also describes a transcranial MR and ultrasound (US) guided Histotripsy (tcMR-USgHt) system. This tcMR-USgHt system can be configured to produce co-registered MRI and US treatment guidance and monitoring at high spatial and temporal resolution and at all intracranial locations.

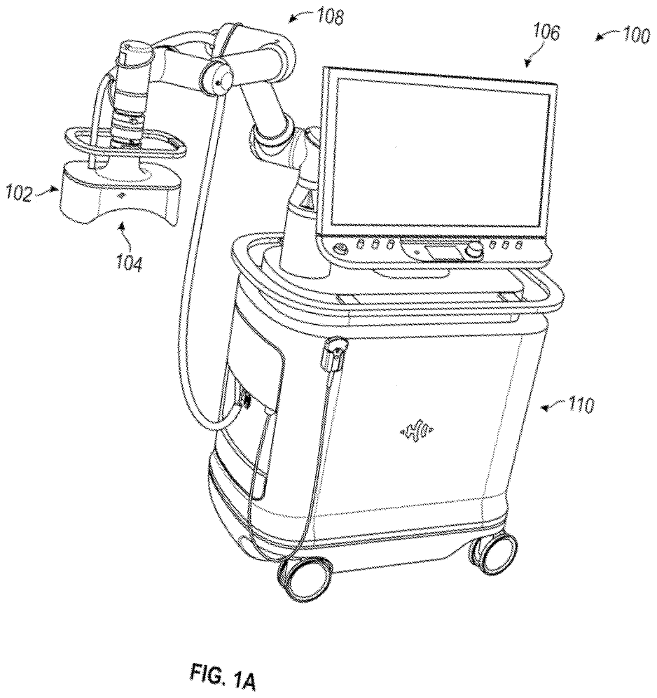

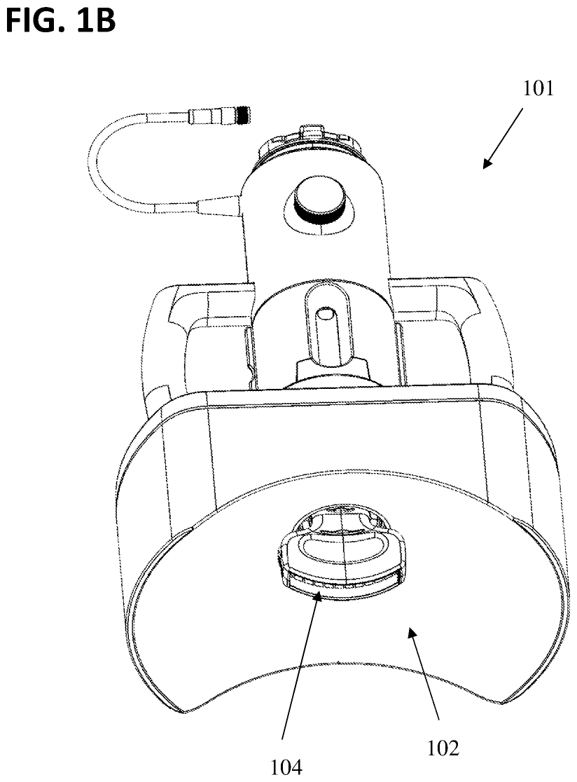

FIG. 1A generally illustrates histotripsy system 100 according to the present disclosure, comprising a therapy transducer 102, an imaging system 104, a display and control panel 106, a robotic positioning arm 108, and a cart 110. The system can further include an ultrasound coupling interface and a source of coupling medium, not shown.

FIG. 1B is a bottom view of a treatment head 101 including the therapy transducer 102 and the imaging system 104. As shown, the imaging system can be positioned in the center of the therapy transducer. However, other embodiments can include the imaging system positioned in other locations within the therapy transducer, or even directly integrated into the therapy transducer. In some embodiments, the imaging system is configured to produce real-time imaging at a focal point of the therapy transducer. The system also allows for multiple imaging transducers to be located within the therapy transducer to provide multiple views of the target tissue simultaneously and to integrate these images into a single 3-D image.

The histotripsy system may comprise one or more of various sub-systems, including a Therapy sub-system that can create, apply, focus and deliver acoustic cavitation/histotripsy through one or more therapy transducers, Integrated Imaging sub-system (or connectivity to) allowing real-time visualization of the treatment site and histotripsy effect through-out the procedure, a Robotics positioning sub-system to mechanically and/or electronically steer the therapy transducer, further enabled to connect/support or interact with a Coupling sub-system to allow acoustic coupling between the therapy transducer and the patient, and Software to communicate, control and interface with the system and computer-based control systems (and other external systems) and various Other Components, Ancillaries and Accessories, including one or more user interfaces and displays, and related guided work-flows, all working in part or together. The system may further comprise various fluidics and fluid management components, including but not limited to, pumps, valve and flow controls, temperature and degassing controls, and irrigation and aspiration capabilities, as well as providing and storing fluids. It may also contain various power supplies and protectors.

As described above, the histotripsy system may include integrated imaging. However, in other embodiments, the histotripsy system can be configured to interface with separate imaging systems, such as C-arm, fluoroscope, cone beam CT, MRI, etc., to provide real-time imaging during histotripsy therapy. In some embodiments, the histotripsy system can be sized and configured to fit within a C-arm, fluoroscope, cone beam CT, MRI, etc.

### Cart

The Cart 110 may be generally configured in a variety of ways and form factors based on the specific uses and procedures. In some cases, systems may comprise multiple Carts, configured with similar or different arrangements. In some embodiments, the cart may be configured and arranged to be used in a radiology environment and in some cases in concert with imaging (e.g., CT, cone beam CT and/or MRI scanning). In other embodiments, it may be arranged for use in an operating room and a sterile environment for open surgical or laparoscopic surgical and endoscopic application, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., anesthesia cart, laparoscopic tower, surgical robot, endoscope tower, etc.).

The Cart may also work with a patient surface (e.g., table or bed) to allow the patient to be presented and repositioned in a plethora of positions, angles and orientations, including allowing changes to such to be made pre, peri and post-procedurally. It may further comprise the ability to interface and communicate with one or more external imaging or image data management and communication systems, not limited to ultrasound, CT, fluoroscopy, cone beam CT, PET, PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri and/or post histotripsy) and to provide access to and display of patient medical data including but not limited to laboratory and historical medical record data.

In some embodiments one or more Carts may be configured to work together. As an example, one Cart may comprise a bedside mobile Cart equipped with one or more Robotic arms enabled with a Therapy transducer, and Therapy generator/amplifier, etc., while a companion cart working in concert and at a distance of the patient may comprise Integrated Imaging and a console/display for controlling the Robotic and Therapy facets, analogous to a surgical robot and master/slave configurations.

In some embodiments, the system may comprise a plurality of Carts, all slave to one master Cart, equipped to conduct acoustic cavitation procedures. In some arrangements and cases, one Cart configuration may allow for storage of specific sub-systems at a distance reducing operating room clutter, while another in concert Cart may comprise essentially bedside sub-systems and componentry (e.g., delivery system and therapy).

One can envision a plethora of permutations and configurations of Cart design, and these examples are in no way limiting the scope of the disclosure.

### Histotripsy

Histotripsy comprises short, high amplitude, focused ultrasound pulses to generate a dense, energetic, “bubble cloud”, capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat cold or ionizing (high) energy to treat tissue. Instead, histotripsy uses acoustic cavitation generated at the focus to mechanically effect tissue structure, and in some cases liquefy, suspend, solubilize and/or destruct tissue into sub-cellular components.

Histotripsy can be applied in various forms, including: 1) Intrinsic-Threshold Histotripsy: Delivers pulses with a 1-2 cycles of high amplitude negative/tensile phase pressure exceeding the intrinsic threshold to generate cavitation in the medium (e.g., ~24-28 MPa for water-based soft tissue), 2) Shock-Scattering Histotripsy: Delivers typically pulses 3-20 cycles in duration. The shockwave (positive/compressive phase) scattered from an initial individual microbubble generated forms inverted shockwave, which constructively interfere with the incoming negative/tensile phase to form high amplitude negative/rarefactional phase exceeding the intrinsic threshold. In this way, a cluster of cavitation microbubbles is generated. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) Boiling Histotripsy: Employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). The application of histotripsy is not limited to a transdermal approach but can be applied through any means that allows contact of the transducer with tissue including open surgical laparoscopic surgical, percutaneous and robotically mediated surgical procedures. It may be further targeted, planned, directed and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism”.

This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When ultrasound pulses less than 2 cycles are applied, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism”.

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6 dB beam width of a transducer may be generated.

With high-frequency Histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically <2 cycles and having a frequency between 100 kHz and 1 MHz) can be applied together with a high-frequency “probe” pulse (typically <2 cycles and having a frequency greater than 2 MHz, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P-level for a region of interest (ROI), while the high-frequency pulse, which provides more precision, can pin-point a targeted location within the ROI and raise the peak negative pressure P-above the intrinsic threshold. This approach may be referred to as “dual frequency”, “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Therapy Components

The Therapy sub-system may work with other sub-systems to create, optimize, deliver, visualize, monitor and control acoustic cavitation, also referred to herein and in following as “histotripsy”, and its derivatives of, including boiling histotripsy and other thermal high frequency ultrasound approaches. It is noted that the disclosed inventions may also further benefit other acoustic therapies that do not comprise a cavitation, mechanical or histotripsy component. The therapy sub-system can include, among other features, an ultrasound therapy transducer and a pulse generator system configured to deliver ultrasound pulses into tissue.

In order to create and deliver histotripsy and derivatives of histotripsy, the therapy sub-system may also comprise components, including but not limited to, one or more function generators, amplifiers, therapy transducers and power supplies.

The therapy transducer can comprise a single element or multiple elements configured to be excited with high amplitude electric pulses (>1000V or any other voltage that can cause harm to living organisms). The amplitude necessary to drive the therapy transducers for Histotripsy vary depending on the design of the transducer and the materials used (e.g., solid or polymer/piezoelectric composite including ceramic or single crystal) and the transducer center frequency which is directly proportional to the thickness of the piezo-electric material. Transducers therefore operating at a high frequency require lower voltage to produce a given surface pressure than is required by low frequency therapy transducers. In some embodiments, the transducer elements are formed using a piezoelectric-polymer composite material or a solid piezoelectric material. Further, the piezoelectric material can be of polycrystalline/ceramic or single crystalline formulation. In some embodiments the transducer elements can be formed using silicon using MEMs technology, including CMUT and PMUT designs.

In some embodiments, the function generator may comprise a field programmable gate array (FPGA) or other suitable function generator. The FPGA may be configured with parameters disclosed previously herein, including but not limited to frequency, pulse repetition frequency, bursts, burst numbers, where bursts may comprise pulses, numbers of pulses, length of pulses, pulse period, delays, burst repetition frequency or period, where sets of bursts may comprise a parameter set, where loop sets may comprise various parameter sets, with or without delays, or varied delays, where multiple loop sets may be repeated and/or new loop sets introduced, of varied time delay and independently controlled, and of various combinations and permutations of such, overall and throughout.

In some embodiments, the generator or amplifier may be configured to be a universal single-cycle or multi-cycle pulse generator, and to support driving via Class D or inductive driving, as well as across all envisioned clinical applications, use environments, also discussed in part later in this disclosure. In other embodiments, the class D or inductive current driver may be configured to comprise transformer and/or auto-transformer driving circuits to further provide step up/down components, and in some cases, to preferably allow a step up in the amplitude. They may also comprise specific protective features, to further support the system, and provide capability to protect other parts of the system (e.g., therapy transducer and/or amplifier circuit components) and/or the user, from various hazards, including but not limited to, electrical safety hazards, which may potentially lead to use environment, system and therapy system, and user harms, damage or issues.

Disclosed generators may allow and support the ability of the system to select, vary and control various parameters (through enabled software tools), including, but not limited to those previously disclosed, as well as the ability to start/stop therapy, set and read voltage level, pulse and/or burst repetition frequency, number of cycles, duty ratio, channel enabled and delay, etc., modulate pulse amplitude on a fast time-scale independent of a high voltage supply, and/or other service, diagnostic or treatment features.

In some embodiments, the Therapy sub-system and/or components of, such as the amplifier, may comprise further integrated computer processing capability and may be networked, connected, accessed, and/or be removable/portable, modular, and/or exchangeable between systems, and/or driven/commanded from/by other systems, or in various combinations. Other systems may include other acoustic cavitation/histotripsy, HIFU, HITU, radiation therapy, radiofrequency, microwave, and cryoablation systems, navigation and localization systems, open surgical, laparoscopic, single incision/single port, endoscopic and non-invasive surgical robots, laparoscopic or surgical towers comprising other energy-based or vision systems, surgical system racks or booms, imaging carts, etc.

In some embodiments, one or more amplifiers may comprise a Class D amplifier and related drive circuitry including matching network components. Depending on the transducer element electric impedance and choice of the matching network components (e.g., an LC circuit made of an inductor L1 in series and the capacitor C1 in parallel), the combined impedance can be aggressively set low in order to have high amplitude electric waveform necessary to drive the transducer element. The maximum amplitude that Class D amplifiers is dependent on the circuit components used, including the driving MOSFET/IGBT transistors, matching network components or inductor, and transformer or autotransformer, and of which may be typically in the low kV (e.g., 1-3 kV) range.

Therapy transducer element(s) are excited with an electrical waveform with an amplitude (voltage) to produce a pressure output sufficient for Histotripsy therapy. The excitation electric field can be defined as the necessary waveform voltage per thickness of the piezoelectric element. For example, because a piezoelectric element operating at 1 MHz transducer is half the thickness of an equivalent 500 kHz element, it will require half the voltage to achieve the same electric field and surface pressure.

The Therapy sub-system may also comprise therapy transducers of various designs and working parameters, supporting use in various procedures (and procedure settings). Systems may be configured with one or more therapy transducers, that may be further interchangeable, and work with various aspects of the system in similar or different ways (e.g., may interface to a robotic arm using a common interface and exchange feature, or conversely, may adapt to work differently with application specific imaging probes, where different imaging probes may interface and integrate with a therapy transducer in specifically different ways).

Therapy transducers may be configured of various parameters that may include size, shape (e.g., rectangular or round; anatomically curved housings, etc.), geometry, focal length, number of elements, size of elements, distribution of elements (e.g., number of rings, size of rings for annular patterned transducers), frequency, enabling electronic beam steering, etc. Transducers may be composed of various materials (e.g., piezoelectric, silicon, etc.), form factors and types (e.g., machined elements, chip-based, etc.) and/or by various methods of fabrication of.

Transducers may be designed and optimized for clinical applications (e.g., abdominal tumors, peripheral vascular disease, fat ablation, etc.) and desired outcomes (e.g., acoustic cavitation/histotripsy without thermal injury to intervening tissue), and affording a breadth of working ranges, including relatively shallow and superficial targets (e.g., thyroid or breast nodules), versus, deeper or harder to reach targets, such as central liver or brain tumors. They may be configured to enable acoustic cavitation/histotripsy under various parameters and sets of, as enabled by the aforementioned system components (e.g., function generator and amplifier, etc.), including but not limited to frequency, pulse repetition rate, pulses, number of pulses, pulse length, pulse period, delays, repetitions, sync delays, sync period, sync pulses, sync pulse delays, various loop sets, others, and permutations of. The transducer may also be designed to allow for the activation of a drug payload either deposited in tissue through various means including injection, placement or delivery in micelle or nanostructures.

### Histotripsy Transducer Design

Systems and methods for implementing miniaturized, endocavity and laparoscopic histotripsy transducers for volume ablation are provided. These systems and methods enable histotripsy treatment in regions of the body with limited acoustic access. A novel, dual material, flat-front acoustic lens design and method provided. Design methods provided can be used to construct flat-front histotripsy transducers, which show increased focusing capability compared to traditional lens construction techniques. These devices demonstrate feasibility for low-frequency, gel-coupled histotripsy transducers. Improvements necessary for clinical translation of such devices are provided, including laparoscopic sized transducers, incorporation of real-time ultrasound imaging, and endocavity phased array development.

Additionally, transcutaneous phased array designs for large volume ablations are provided. In some examples, a modular, kerf-minimizing approach to therapeutic ultrasound phased array construction is implemented on a phased array system designed for treating the human abdominal region. Some embodiments of the resulting phased array can feature up to 92% aperture utilization (previous modular construction techniques: 50-60%), and be configured to generate peak negative focal pressures in excess of 100 MPa. Also, a novel, semi-cylindrical phased array design for the treatment of soft tissue sarcoma is provided. The novel geometry enables several promising rapid ablation techniques, including multi-focal treatment and a focus enlarging technique resulting in a predicted focal volume larger than a typical focal volume.

This disclosure provides endocavity or laparoscopic histotripsy transducers focused on minimizing aperture size, while maintaining the necessary transducer specifications to allow for clinically relevant ablation rates and transducer performance (working distance, etc.). Specifically, systems and methods provided herein are configured to decrease operating frequency (≤1.5 MHz), while maintaining an aperture size suitable for transrectal or transvaginal operation (<4 cm aperture).

### Transcutaneous Histotripsy Phased Array Focus

This disclosure provides fabrication techniques to enable modular, high aperture utilization fabrication methods. These methods can be used in designing and fabricating high power phased arrays for rapid ablation techniques, as well as other advanced histotripsy treatment capabilities such as AC and passive acoustic mapping (PAM). Phased arrays are also provided specific to the treatment of liver cancer and STS. As the anatomy surrounding these indications varies substantially, the instrumentation design is optimized to allow for efficient treatment of these locations. Additionally, the effect of treatment parameters such as EFS point spacing, focal pressure, and EFS sequence on ablation rate are explored using current generation instrumentation. This provides a deeper understanding of the effects of these parameters, as well as enabling rapid, large volume treatment. Through this optimization, ablation rates in the range of >2 cubic centimeters per minute are achievable.

This disclosure provides an endocavity histotripsy transducer with a low frequency (≈1 MHz) and capability for volume ablation at a reasonable speed towards practice in the clinical settings. A clinical treatment rate can be in the range of 1 ml per 10 minutes of treatment, with higher ablation rates needed for larger volume targets. This transducers disclosed herein provide capabilities for range of applications, such as ablation of the prostate or chronic pelvic abscesses. Use of an endocavity histotripsy transducer for treatment of these applications can bypass the limited acoustic window (due to the pelvic bones and bowel or other obstructions in tissue). In order to perform endocavity histotripsy, the transducer is configured to produce peak negative focal pressures of up to 30 MPa or more. Additionally, a working distance between 10 and 40 mm is provided. In some embodiments, the transducer can have a similar size to existing transrectal HIFU transducers (approximately 35 mm aperture). In some implementations, the transducer can be constructed inexpensively (approximately $200 in material cost per transducer), in a relatively simple and reproducible way (no more than a few hours fabrication time, no highly specialized skills required). This allows the transducer to be treated as semi-disposable (requiring only moderate durability), and would allow for easy replacement if damaged.

This disclosure provides the design, fabrication and characterization process for endocavity histotripsy transducers. First, piezoelectric materials, matching layer materials and drivers can be modeled and selected in order to determine a material and driver combination best suited for achieving high focal pressure. These results guided the design of the endocavity transducer.

While this disclosure provides miniaturized histotripsy transducers, there are additional features that can be included for clinical use. Most prominently, an ultrasound imaging guidance system can be integrated into the design in order to enable real-time targeting and treatment by clinicians or researchers. In some embodiments, this can be a transducer that fits around an existing transrectal or transvaginal imaging probe, or a custom fabricated low profile imaging transducer integrated directly into the transducer scaffold. For example, an imager of approximately 8×20 mm aperture size could be incorporated into transducer described above, while still allowing the transducer to generate 30 MPa peak negative pressure. The therapeutic transducer could be co-registered with the imaging transducer, providing pre-treatment targeting and visual feedback throughout the treatment process.

Additionally, for in-vivo use, a coupling mechanism must be implemented in order to ensure maximum transmission of sound from the transducer to the treatment zone. This could comprise a fluid filled bladder or balloon covering the face of the transducer. In the case of prostate treatment, this bladder or balloon would be configured to provide a surface that would conform to the rectal wall and allow transrectal sound transmission to the prostate.

### Flat Front Dual Material Acoustic Lenses for Histotripsy Transducers

Performing histotripsy treatments with large concave ultrasound transducer arrays currently requires use of a cumbersome water bath coupling system. The water bath coupling system include a large water bolus placed on the patient, which the histotripsy transducer is placed into, allowing sound to be transmitted to the body through the water bath. While this coupling system has been used with success extensively, it is a clinical barrier to the acceptance of histotripsy as a treatment modality, as it is difficult to use, and creates potential challenges during treatment if the patient needs to be repositioned. This disclosure provides ultrasound transducers for histotripsy that are designed to have a flat or slightly convex front face, thereby allowing for acoustic coupling directly to a patient's skin with standard ultrasound gel, similar to how ultrasound imaging probes are used.

Water bath coupling is the standard coupling method for histotripsy due to the size and geometry of the transducers used. To generate the high amplitude focal pressures necessary for histotripsy, transducers are typically large, and utilize a concave spherical shell geometry to generate high focal gain. Most often, multiple piezoelectric elements are arranged over this spherical shell geometry. Sometimes each element is bonded to an acoustic lens to further increase focal gain, while other transducers are designed as phased arrays, capable of performing electronic focal steering and phase aberration correction. Typically, concave lenses fabricated out of high sound speed (>1500 m/s) materials are used for histotripsy. It is possible to make convex acoustic lenses, if a material with a sound speed lower than the target media is used. This approach is not commonly used for histotripsy transducers, as most materials with sound speeds lower than that of biologic tissue (≈1500 m/s) are also attenuative, which lessens their usefulness for high amplitude applications.

As an alternative to the concave geometries previously described, flat, electronically-steered phased arrays have been developed for HIFU treatment. HIFU, as opposed to histotripsy, uses intermediate amplitude, long acoustic exposures to generate tissue necrosis via heating, while Histotripsy is an entirely non-thermal therapy modality. Thus, transducer engineering requirements are very different between transducers designed for HIFU and histotripsy. However, current material performance limitations for histotripsy are such that the requisite focal pressures would be unattainable for such an array. Due to these limitations, this disclosure provides lens-based alternatives to create a flat-front histotripsy transducer capable of producing the focal pressures required to generate cavitation in tissue.

Traditional acoustic lenses are made out of a single material of higher or lower sound speed than the target media. When designing a lens material with a higher sound speed, a concave elliptical shape is used in accordance with Fermat's principle of least time, resulting in equal time-of-flight (tof) between the focus and all points on the face of the transducer. When a low sound speed lens material is used, a convex shape is formed for the same effect. Alternatives to traditional single material elliptical lenses include Fresnel zone plate (FZP) and phase-continuous Fresnel lenses. FZPs are binary masks, which work by effectively blocking portions of the aperture that will arrive at the intended focal location out of phase with the rest of the transducer, eliminating any destructive interference from occurring at the focal location. While this can result in a substantial pressure amplitude increase, it is inefficient, as approximately half of the aperture is blocked from contributing to the pressure field. Phase-continuous Fresnel lenses are designed by removing the thickness of a standard acoustic lens in a staircase shape, where the thickness of each step is an integer multiple of the wavelength, allowing sound from any point on the source arrive at the focus in phase. While this approach solves the efficiency problems of the FZP, it does not allow for a flat-front design as it is currently implemented.

This disclosure provides a flat-front acoustic lens that uses two materials, one of high sound speed (>1500 m/s), and one of lower sound speed (<1500 m/s). The high sound speed material can be used to construct a concave lens structure, which is then filled with a low sound speed material, allowing for the lens to have a flat front profile. The profile of the surface between the two lenses is designed specifically to account for the speed of sound of each material, as well as the speed of sound of the target media, as these parameters affect the time of flight (tof) for a ray of sound from any point on the transducer to the intended focal location. Additionally, the angle of refraction at each boundary will affect the tof, and is accounted for in each design.

This disclosure describes the design, fabrication, and characterization of three transducers. The first is a transducer based on a standard, single material, concave elliptical lens. The second is a transducer based on the previously described flat-front design, using a high sound speed lens material, and a low sound speed fill material. The third transducer is a modified flat-front design to use the concept of phase-continuous Fresnel lenses, allowing the lens to be much profile than the other designs.

### Lens Design Algorithm

Traditional, single material acoustic lenses are well understood, and their curvature follows a straightforward calculation. This description of curvature, however, will not correctly describe the lens profile for the desired, flat-front, dual-material lens desired here. This is due to the additional “fill” or low sound speed material used to create the planar coupling surface. The inclusion of this additional layer results in an additional boundary between the acoustic source and the focal point, meaning there is additional refraction through this layer, affecting the acoustic path length, and tof between the source and focal point. This effect could be mitigated by using a fill material with a speed of sound equal to that of the media the sound is being focused in, however, this limits the range of suitable materials that can be used as a fill material. Because of this, an alternative approach to determining the lens profile is necessary. To calculate this profile, a simple ray-tracing approach can be used, in which the lens profile is determined such that the tof between the source and the focal point is equal for any radial distance across the transducer. The following algorithm determines the profile of the boundary between the two lens materials, assuming the front face of the lens to be flat. Additionally, it allows for Fresnel-based “phase-wrapping” to be used to decrease the overall thickness of the lens.

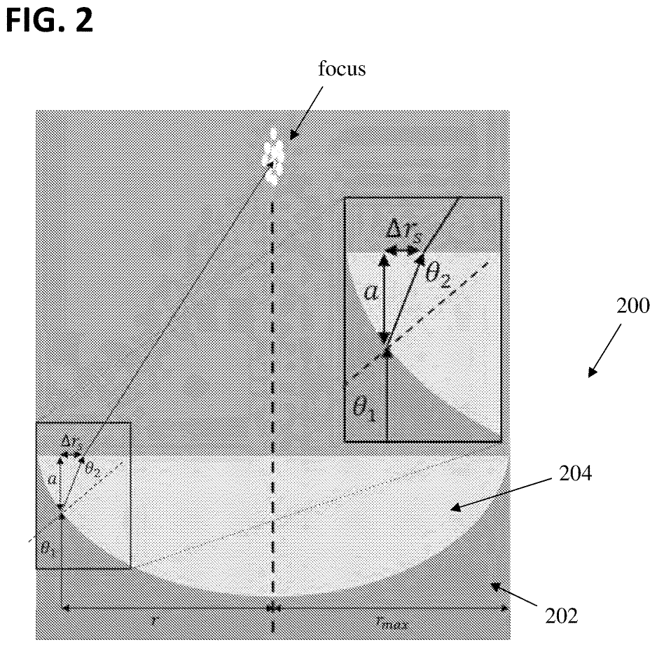

FIG. 2 provides a graphical representation of flat front profile determination parameters for a flat profile lens 200 having a first material 202 and a second material 204. As described above, the first material 202 can have a high sound speed (>1500 m/s) and a concave shape, and a second material 202 of lower sound speed (<1500 m/s) used as a fill material.

First, the overall thickness of the lens is calculated. Referring to FIG. 2, this can be done by solving for the thickness such that the tof for the center (Equation 1) of the lens to the focus is equal to that from the edge of the lens (Equation 2) to the focus. Equation 2 is obtained by recognizing that there will be no fill material at the edge of the transducer, and factors in the phase correction when designing a Fresnel lens. [symbol] ₚₘᵢₙ is the minimum thickness of the high sound speed material 202, cₚ is the speed of sound in the high sound speed material, [symbol] ₛₘₐₓ is the maximum thickness of the low sound speed material, cₛ is the speed of sound in the low sound speed material, [symbol] _{w} is the distance from the water-low sound speed material boundary to the desired focal position, c_{w} is the speed of sound in water, rₘₐₓ is the maximum radius of the transducer, n_{seg} is the number of Fresnel segments desired on the lens, and P is the period of the sound being transmitted through the lens.

$${tof}_{center} = \frac{{y}_{pmin}}{{c}_{p}} + \frac{{y}_{smax}}{{c}_{s}} + \frac{{y}_{w}}{{c}_{w}} \qquad ( 1 )$$

$${tof}_{edge} = \frac{{y}_{pmin} + {y}_{smax}}{{c}_{p}} + \frac{{y}_{w}^{2} + {r}_{max}^{2}}{{c}_{w}} - {n}_{seg} P \qquad ( 2 )$$

After solving for the thickness of low sound speed material in the lens, a similar ray-tracing approach can be used determine the profile between the low sound speed material and high sound speed material. In order to perform these calculations, the lens is constrained to have a constant overall thickness across all possible radii, but allows the thicknesses of the first and second materials to vary. The center position in the lens is initialized to have the minimum high sound speed material thickness ( [symbol] ₚₘᵢₙ) and the maximum low sound speed material thickness ( [symbol] ₛₘₐₓ). The lens profile is then calculated using a numerical approach. For the i-th radial point, the calculation is as follows.

$${tof}_{p} = \frac{ypr}{{c}_{p}} \qquad ( 3 )$$

$${tof}_{s} = \frac{\overline{{a}^{2} + Δ {r}^{2}}}{{c}_{{s}_{s}}} \qquad ( 4 )$$

$${tof}_{w} = \begin{matrix}{y}_{\underline{w}}^{2} + {( r - Δ {r}_{s} )}^{2} \\ {c}_{w}\end{matrix} \qquad ( 5 )$$

$${tof}_{r} = {tof}_{p} + {tof}_{s} + {tof}_{w} \qquad ( 6 )$$

$$Where$$

$$a = ( {y}_{smax} + {y}_{pmin} - ypr ) \qquad ( 7 )$$

$$Δ {r}_{s} = a tan ( {θ}_{1} - {θ}_{2} ) \qquad ( 8 )$$

$$θ = a tan ( \frac{ypr - {ypr}_{i - 1}}{Δ {r}_{1}} ) \qquad ( 9 )$$

$$θ = a sin ( \frac{{c}_{s} * sin ( {θ}_{1}}{{c}_{p2}} ) \qquad ( 10 )$$

Where Δr is the step size between adjacent radial points used for the profile calculation, and [symbol] prᵢ₋₁ is the profile determined in the previous step (Δr closer to the center of the lens). A graphical description of the parameters is given in FIG. 2. By solving for a profile resulting in a tof equal to that for the center and edge cases, one can determine the profile for the dual-material, flat-front lens. If a Fresnel lens is desired, a few extra steps need to be taken. First, when checking for equality between Eq. 1, 2, and 6, if no profile is found such that all expressions are equal, a new “wrap” must be started. Due to the approximations used to calculate θ₁ and θ₂, two points must be initialized at the beginning of each new wrap. The two points were initialized as follows, where [symbol] prᵢ is the first point in the new wrap, and yprᵢ₊₁ is the second point in the new wrap.

$${ypr}_{i} = {y}_{smax} + {y}_{pmin} - ( {c}_{p} - {c}_{s} ) * P \qquad ( 11 )$$

$${ypr}_{i + 1} = {ypr}_{i} + ypri - 1 - {ypr}_{i - 2} \qquad ( 12 )$$

While this initialization is very close to providing ideal tof, there is an error associated with refraction through the low sound speed layer (due to the adjusted thickness). Because of this error, a simple error loop correction can be implemented to ensure the tof error is kept small (10 ns).

While this initialization is very close to providing ideal tof, there is an error associated with refraction through the silicone layer (due to the adjusted thickness). Because of this error, a simple error loop correction was implemented to ensure the tof error was kept small (10 ns). After determining the profile, a spline was fit to the profile for use in simulations and CAD software.

The resulting lens profiles for the concave/elliptical, flat-front dual material, and

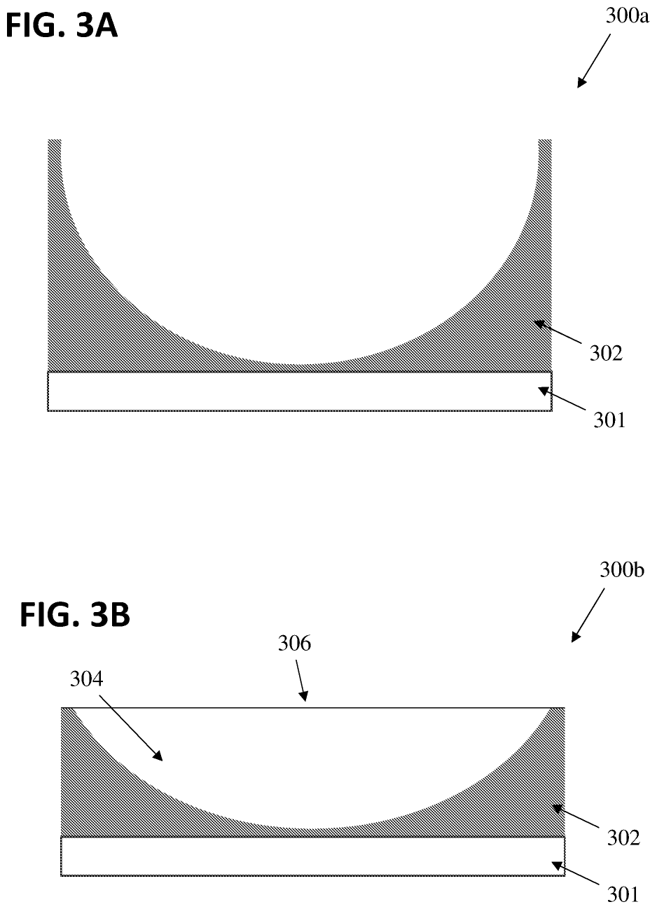

flat-front Fresnel dual material lenses can be seen in FIGS. 3A-3C. FIG. 3A shows a lens 300a having a concave shape made out of a high sound speed material 302 (e.g., >1500 m/s). FIG. 3B, however, shows a flat-front dual material lens 300b that comprises a first material 302 of a high sound speed (e.g., >1500 m/s) and a second material 304 of a low sound speed (e.g., <1500 m/s). The lens 300b is significantly thinner than the lens 300a, and further provides a flat transmitting surface 306. As shown, the lens 300b is also flat at the interface between the high sound speed material 302 and the transducer array 301 of one or more transducer elements, enabling a flat transducer array and flat emitting surface for direct coupling to a subject.

FIG. 3C shows an embodiment of a flat-front Fresnel dual material lens 300c that comprises a first material 302 of a high sound speed (e.g., >1500 m/s) and a second material 304 of a low sound speed (e.g., <1500 m/s). The lens 300b is thinner than both lens 300a and 300b, and further provides a flat transmitting surface 306. Fresnel steps 308 are formed in the first material. Furthermore, the lens 300c can include a concave portion 310 centrally located in the lens, with the Fresnel steps 308 positioned peripherally to the concave portion.

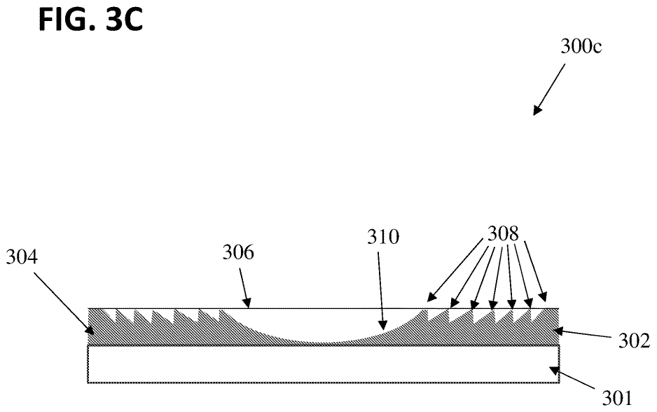

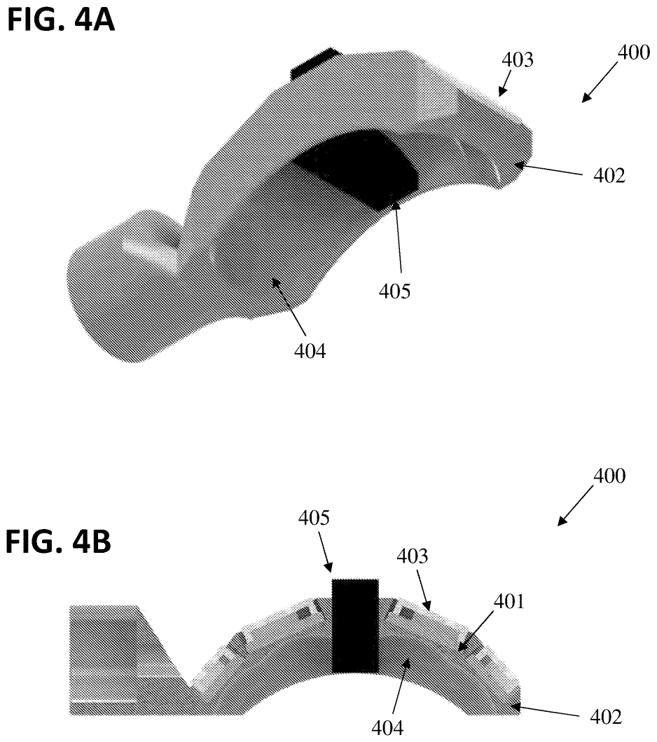

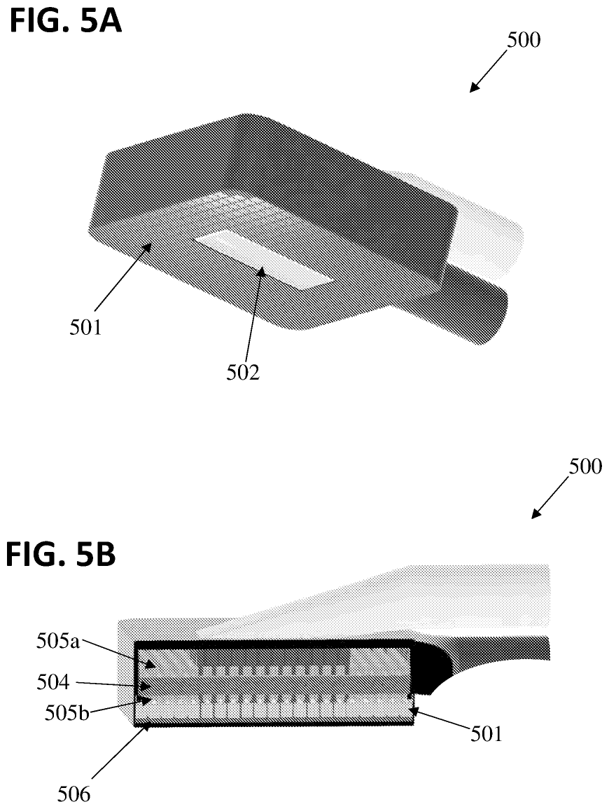

Any of the lenses described above can be affixed or acoustically coupled to one or more transducer elements 301, or arrays of transducer elements 301, to provide flat-front, low-profile histotripsy transducer probes or arrays configured to produce pressures in tissue capable of generating histotripsy cavitation while allowing for direct coupling of the transducer array or probe to the skin or target tissue of a subject (or with the use of a standard acoustic coupling gel or medium). The transducer elements 301, lenses 300a/b/c, and any additional electrical connections or components described herein can be incorporated or disposed within a transducer array or probe housing, such as is shown in FIG. 1B or any other embodiment herein (e.g., the probes of FIGS. 4A-4B or 5A-5B).

In some embodiments, any of the embodiments described herein and including the dual-material acoustic lens can further include one or more ultrasound imaging elements incorporated into the transducer and/or lens, such as a centrally located ultrasound imaging transducer or array (e.g., as shown in FIG. 5A).

Due to the use of the second, low sound speed material, the thickness of the compound lenses provided above is greatly reduced compared to the elliptical lens of FIG. 3A. In one specific embodiment, the elliptical lens is 23 mm thick, but the compound lens in FIG. 3B is reduced to 12.8 mm or less. By using the phase-wrapping technique on the Fresnel lens of FIG. 3C, the thickness can be further reduced to 3.7 mm or less. The flat-front dual material lenses and elliptical single material transducers are capable of generating cavitation with histotripsy pulses when paired with ultrasound transducer elements and electrically coupled to histotripsy pulse generators and amplifiers as discussed herein.

The key advantage of the flat-front lenses described herein is their ability to be dry-coupled to target media. Typically, focused ultrasound transducers require the use of a cumbersome water bath coupling mechanism to acoustically couple the sound from the transducer to the target tissue. The water bath requirement is due to the spherical or elliptical geometry of the focused ultrasound transducers or lenses. By developing a flat-front acoustic lens, a tightly focused sound field can be created without the use of a concave lens or spherically focused transducer. This significantly decreases the difficulty of use of these transducers, and enables easier operation, especially in some treatment environments, such as endoscopic histotripsy.

For some therapeutic ultrasound techniques, such as histotripsy, short pulses (no more than a few acoustic cycles) are desired. In these cases, a phased array approach will be beneficial in developing larger transducers than the ones presented in this study. As the aperture increases in size, so does the thickness of the lens (for a given working distance). As the lens materials are attenuative, it is desirable to maintain a thin lens. For large transducers, maintaining a thin lens will require the use of the Fresnel-based design. The resulting lens will have many phase wraps, necessitating the use of a longer tone-burst driving pulse than would be desired, unless a phased array approach is used. By using a Fresnel-assisted phased array, single cycle pulsing can be used by phasing each element to account for its Fresnel phase wrap number, as well as any desired phasing for EFS.

An added benefit of this approach is the decrease in lens thickness. Especially in the case of the Fresnel based design, the overall thickness of the transducer is reduced dramatically. For some applications of therapeutic ultrasound, such as endoscopic or laparoscopic treatments, lower profile transducers can be an advantage, as they allow the transducer to be more easily navigated to a target region.

In some embodiments, the high sound speed material can be a 3D printed plastic housing, and the low sound speed material can be a silicon fill material. The design in this embodiment requires only a single piezoelectric element, a silicone fill material, and a simple 3D printed housing to be constructed. For this reason, this embodiment may be of interest for a variety of fields requiring focused ultrasound transducers that would benefit from dry coupling. For example, a similar transducer could be used to conduct Resonant Acoustic Rheometry, easing the coupling requirements, while maintaining simple transducer construction and driving electronics. Another example would be use of a dry-coupled transducer for sonothombolysis, as an alternative extracorporeal transducer for micro-bubble and nano-droplet mediated sonothrombolysis.

Laparoscopic Single Focus Transducer with Imaging

For practical clinical use, endocavity and laparoscopic therapy transducers can include an integrated guidance system to give clinicians real-time feedback about where therapy is being applied. The most straightforward approach to guidance is integration of a b-mode ultrasound imaging probe into the therapy probe or array. As the main limitation on endocavity and laparoscopic histotripsy probes is size, the imaging probe can be very low profile, as to not take up a large amount of the available aperture space. While many commercially available imaging probes have suitable (small) active aperture sizes, they typically also feature large housings designed for ergonomics rather than to be low profile. For this reason, the integrated imaging probe can be custom designed and fabricated for incorporation with the endocavity histotripsy transducer.

In one embodiment, the design specifications include a desired working distance in the range of 10-15 mm, with the maximum width of the transducer not to exceed 20 mm.

Fabrication of the transducer can be very similar to the transducer described above. FIGS. 4A-4B shows one example of a laparoscopic histotripsy transducer probe 400 that includes a concave housing 402 that incorporates lenses 404 into the housing and provides electrical insulation for ultrasound transducer elements 401. Backing clamp pieces 403 are positioned on the back side of the transducer elements 401. An integrated imaging probe 405 is shown extending through a central portion of the housing 402 towards the transmitting surface of the probe.

In some embodiments, the transducer housing and backing clamp pieces can be printed out of Vero Clear (c≈2500 m, ρ≈1200 kg) material, on a 3D printer, and piezoelectric elements can be water jet cut from stock pieces of DL-53 piezocomposite. Backing clamp pieces can be threaded onto micro-coaxial cable, and soldered to the piezoelectric elements. Other suitable materials can be used. Elements can then be epoxied into the housing using epoxy. Clamping pressure may be applied via the backing clamp pieces, to allow the transducer to cure.

### Flat Endocavity Array

While many studies have been successfully performed using single focus histotripsy transducers, phased array transducers offer significant advantages in many respects, including the ability to perform EFS, and PAM or passive cavitation imaging (PCI). Depending on the particular indication, the ability to perform one or more of these techniques may be critical for clinical adoption. For example, for an especially large volume target, such as a pelvic abscess, which can reach volumes greater than 300 milliliters, EFS may be needed for practical clinical adoption as it would enable a large volume to be ablated with less mechanical scanning. For other applications requiring precise treatment dosing, the ability to receive acoustic cavitation emission (ACE) signals may be of significant importance in the future. As such, it is necessary to evaluate the possibility of developing an endocavity phased array. This disclosure presents the design, simulation, and initial testing for fabricating such a transducer.

Specifically, a novel, 2D phased array design is provided. This design allows for a high degree of steerability, allowing for very large ablations in relation to the aperture size of the transducer. The aperture size is such that the transducer would be appropriate for transrectal or transvaginal use, and could be applicable to a wide range of indications. Additionally, the 2D design offers significant fabrication benefits, allowing for transducers to remain relatively easy and inexpensive to fabricate.

### Flat Array Histotripsy Probes and Fabrication Methods

Significant challenge is associated with miniaturization traditional, geometrically focused histotripsy transducer rapid prototyping techniques. These challenges become especially apparent at the elemental size range required for endocavity histotripsy phased arrays (<3 mm). As such, new fabrication techniques needed. These fabrication techniques will not only push endocavity and endoscopic transducer technology forward, but also advance histotripsy transducer technology as a whole, as they could be translated to large aperture transducers as well if decreased element size was desired.

A significant benefit of 2D transducer construction is that the fabrication burden as the potential to be significantly lessened. Because the array is arranged two dimensionally, machining operations are simplified greatly, and a traditional dicing saw can be used for all operations. This assumes elements are arranged in a standard grid, and are rectangular in shape. In short, a piezocomposite of the same aperture size as the full array could be bonded to the desired matching layers in one piece. Subsequently, the piezocomposite would be diced into multiple, individual elements while leaving the matching layer intact, resulting in a 2D grid of elements. While the machining operations are simplified, other challenges arise which are associated with creating reliable, high voltage capable, electrical contacts to each element. The electrical contacts will need to avoid arcing between adjacent elements when they are being driven out of phase.

FIGS. 5A-5B illustrate one embodiment of a flat array histotripsy transducer array or probe 500. Referring to FIG. 5A, the probe 500 can include a transducer array 501 comprising a plurality of transducer elements and an optional imaging array 502 comprising one or more imaging transducers positioned within the transducer array 501. As shown in FIG. 5A, the imaging array is centrally positioned in the probe. In one implementation, therapy transducers presented here feature a 15.5×7 mm central bore to allow the imaging probe to be inserted into the probe 500. The imaging probe could feature a 15 MHz center frequency, as to provide high image quality for shallow targets, while not being so high frequency to disable an imaging depth of less than the desired focal length of the therapy transducers.

FIG. 5B shows a cross-sectional view of the probe 500, which includes an array 501 of transducer elements. A printed circuit board (PCB) 504 contains spring pin electrical connectors 505a/b for each of the transducer elements of the array 501. Micro-coaxial cabling (not shown) can be electrically connected to spring pin electrical connectors 504a to provide an electrical connection to the back side of the transducer elements via spring pin electrical connectors 504b. Soldering the electrical connections to the back sides of each element is feasible but burdensome. The through-hole spring contact pins described above can have a maximum diameter of 1.1 mm, and have an operating force of 15-60 grams. A significant advantage of spring contact pins is that a transducer head can be designed to be removable from the probe, while the cable bundle could be reused on another transducer.

Potential for arcing between adjacent elements can also be addressed. Individual elements can be electrically insulated by putting them in an epoxy, however this would also negate the removable advantage the spring pins provide, as the pins would become permanently bonded to the transducer. Alternatively, a high strength dielectric grease (can be used to electrically insulate the elements 501 and spring pins 505b. In some embodiments, the dielectric grease can reduce arcing potential, including when driving elements out of phase with driving voltages up to 4000 Vₚₚ.

In order to create a reliable high voltage electrical connection to the front of the transducer array elements, strategies other than traditional soldering are provided in this disclosure. For the front side of the elements, direct soldering is not feasible, as the piezocomposite is bonded to the matching layer prior to being diced. In order to make electrical connection to this side of the elements, a conductive mesh-epoxy composite matching layer 506 can be used. The matching layer 506 can comprise, for example, a grid or mesh pattern of a conductive material such as copper arranged such that an electrical connection is provided to the front surface of each of the transducer elements in the transducer array 501. The conductive material can be embedded in an epoxy or other suitable material matching layer to provide 1) the electrical connection to the front surface of each transducer element, 2) provide appropriate acoustic matching between the transducer elements and the target tissue or coupling material, and allow for transmission of ultrasound/histotripsy pulses through the conductive material. This matching layer effectively allows elements to be driven to high voltages reliably, while also providing effective acoustic matching to the coupling fluid or patient's skin/tissue.

While the probe of FIGS. 5A-5B is shown with the matching layer and conductive mesh embodiment on or near the flat transmitting surface of the probe, it should be understood that the probe design of FIGS. 5A-5B could also include the dual-material flat front lenses of FIGS. 3B-3C in other embodiments. In additional embodiments, the conductive mesh of FIGS. 5A-5B could be incorporated into the dual-material lenses of FIGS. 3B-3C. For example, the conductive material could be incorporated into the first (elliptical/concave) material of the dual-material acoustic lens, and the second material could be used to create the flat surface matching layer.

For histotripsy treatment, a lower frequency (to a point) is desirable due to larger focal volume, resulting in a faster treatment rate. Additionally, lower frequency is associated with lower attenuation and aberration. For these reasons, a central frequency of 1 MHz and elements of size 1.3 mm may be chosen as a starting point for further optimization.

The method of fabrication described above allows for rapid fabrication of the probe, which can be completed over the course of only a few hours of active fabrication time. The resulting probe can include 36 or more total elements depending on the desired size of the probe.

One area of note is the difference between transducer performance trends of spherically focused arrays in comparison with 2D, flat arrays. For example, with a typical spherically focused array, such as the design presented above, the free-field focal pressure will increase with an increase in frequency, due to a higher focal gain. This is not true of 2D arrays, as an increase in frequency also increases the directivity of each element, meaning the array is less capable of being electronically steered to a focus. This effect also manifests in the effective working distance of the 2D array, where higher frequency arrays are predicted to generate their highest pressure further from the face of the transducer.

The two transducer arrays or probes presented herein require different coupling mechanisms. The laparoscopic transducer uses elements arranged in a spherical geometry, as most histotripsy transducers do. This transducer will therefore require a water balloon or water bath coupling mechanism to be practically used in a laparoscopic environment. A key advantage of the 2D array is that its geometry will allow for a “dry” coupling, using standard ultrasound gel. This negates the need for a water bath or balloon coupling system, which could be advantageous for extracorporeal targets as well.

Another use for the flat array technology for larger, extracorporeal histotripsy. For example, a larger 2D array can be configured for treating large volumes in the abdomen, such as the treatment of liver cancer. Such an array maintains the steerability benefits associated with the flat design, and also has the ability to be coupled to the body with ultrasound gel, rather than using a large water bath coupling system.

Alternatively, flat transducer modules can be constructed as part of a larger, spherically focused phased array system. This allows for fabrication of an array of many elements to be substantially easier, and allows for steerability of the array to be increased substantially in comparison to a design featuring fewer, larger elements. Increasing element quantity may be of interest as applications for transmit-receive capable histotripsy arrays are further developed, as a higher number of elements may allow for higher performance, such as more precise aberration correction, or cavitation localization.

### Soft Tissue Sarcoma Array

Soft-tissue sarcoma (STS) are malignant tumors which develop in the mesenchymal cells, often in the arms or legs. According to American Cancer Society Statistics, approximately 13,000 new patients are diagnosed with STS each year. STS can grow to very large sizes, often exceeding 10 cm in their largest dimension. Additionally, they can encompass nerves and critical vessels. Surgical resection is the first-line treatment; however, resection is often difficult due to the critical structures encompassed by the STS.

It is possible to shrink STS tumors with radiation therapy prior to resection, however, since tumors can be very large, the necessary radiation dose can exceed the range suitable for the particular location of the tumor. Additionally, STS have a high risk of reoccurrence, and radiation is only able to be used once at a particular site. Alternative thermal ablation strategies, such as radio-frequency, cryo, and microwave ablation risk damage to critical structures near the tumor, and thus, are not viable alternative ablation strategies due to the often-encompassed nerves and vessels. HIFU has been used to treat STS in humans and dogs, but bears similar risks to other thermal ablation strategies.

As an alternative to the previously mentioned minimally and non-invasive ablation modalities, histotripsy has potential to be used for ablation of STS, and offers several significant benefits. First, histotripsy is non-invasive, as the therapeutic ultrasound pulses are delivered from outside of the body. Second, histotripsy has been shown to be effective in treating large tissue volumes, which is one of the limitations of thermal ablation modalities. Additionally, histotripsy has been shown to perform tissue selective ablation, meaning nerves and vessels within the STS can be spared from critical damage. Lastly, histotripsy has been shown to lead to local tumor regression and reduced metastases, and has been shown to be safe for treatment of the liver in humans.

To adapt histotripsy for the treatment of STS, progress needs to be made on several fronts. First, as STS tumors can grow to very large sizes, histotripsy systems and treatment strategies need to be developed to allow for rapid treatment of STS tumors. Second, as STS tumors can grow very close to the skin surface, treatment strategies need to be developed to minimize skin damage to patients during treatment. Finally, while standard b-mode ultrasound can be used for initial targeting of the tumor, 3D monitoring techniques need to be developed to allow for treatment monitoring during rapid ablation of the STS tumor.

In some embodiments, a transducer design is optimized for the treatment of STS based on patient data on the size and location of STS tumors.

Most previously designed histotripsy phased arrays (such as those presented above) feature elements arranged over a spherical shell. In typical arrangements (approximately square or round elements, etc.), this will result in comparable lateral steering ranges (i.e. in the X and Y directions), which is suitable for tumors or other masses that are roughly spherical. In the case of STS, tumors tend to be significantly longer in one dimension. An optimal transducer design will take this into account, and will allow for treatment strategies which are designed specifically for the tumor size and shape being targeted.

As STS tumors are approximately cylindrical in shape, a semi-cylindrical transducer aperture shape can be used in some embodiments. This aperture shape allows for three distinct ablation strategies to be tested: 1) traditional EFS, 2) multi-focal EFS, similar to the aforementioned technique, but with multiple, simultaneous foci, spatially separated from each other, and 3) large focus ablation, where the array is specifically phased such to create a long, skinny focus, mirroring the shape of the tumor. Additionally, as semi-cylindrical geometry only curves in one direction, it allows for fabrication advantages over spherically focused transducers.

In comparison to spherically focused arrays, such as the liver array presented above, the semi-cylindrical geometry of the STS array provides significant fabrication benefit. Because the array is only curved in one dimension, flat, multi-element modules are able to be utilized, enabling fabrication methods similar to that used to fabricated flat, 2D phased arrays, such as the proposed endocavity phased array above.

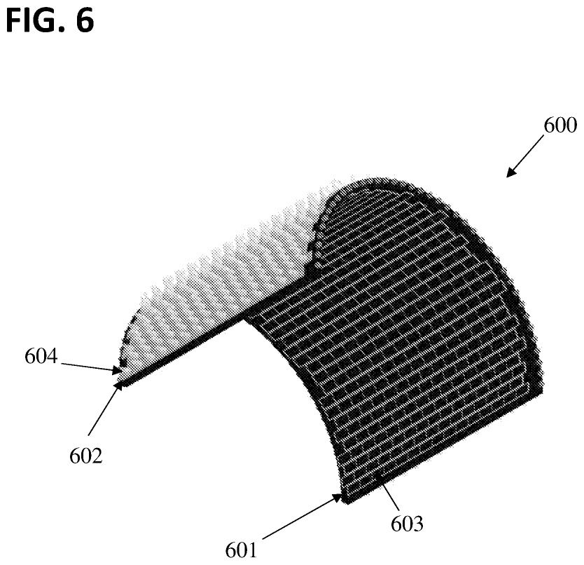

FIG. 6 shows one embodiment of a STS histotripsy array 600. Fabrication of the array may utilize 3D printing technology to manufacture three components of each module. The first is a matching layer with a mating feature on the back, allowing it to be easily mated to the second piece, the module housing. The housing provides mechanical structure for the module, and protection on the sides of the module. The last piece is a lid for the module, which leverages multi-material 3D printing technology to incorporate an elastomeric material to serve as a strain relief for each cable.

To fabricate a module, first, a piezoelectric strip 601 may be bonded to a copper composite strip 602 and 3D printed plastic matching layer stack (positioned in front of the piezoelectric strip). For the array in one embodiment, the piezoelectric strip may have dimensions of 200×7.4 mm, and may have a 3×3 mm section of one corner sanded or cut off to allow electrical access to the copper composite matching layer.

Next, the PZT can be be diced into a plurality of individual elements, such as with a 0.25 mm dicing blade (resulting in a 0.25 mm kerf between elements). The module housing can then then be bonded to the matching layer stack, and a micro-coaxial cable can be threaded through its respective strain relieve, and will be soldered directly to each element at electrical connections 604. The module can then be backfilled with epoxy, and the strain-relief lid can be bonded to the back of the housing, completing the module.

After fabricating the required modules for the array, they can be installed on a scaffold. The scaffold could be printed using a large format 3D printer, or could be slightly re-designed to be machined out of aluminum or another material. The scaffold allows each module to be inserted into a slot, which holds each module into position, including the row offsets discussed previously. A screw may be used on each end of every module to secure the module to the scaffold.

A significant novel aspect of the STS transducer design presented herein is the use of non-standard geometry. Previously, histotripsy transducers have been built around a spherical shell geometry, due to the focal gain achievable when using this arrangement. Due to material advances, even using a relatively conservative surface pressure calibration of 2 MPa, it is now possible to achieve cavitation inducing pressures using non-spherical geometry phased array. Specifically, for the treatment of STS, this provides a key advantage. First, as the transducer geometry mirrors the tumor geometry, the treatment steering range is in alignment with the tumor. This allows EFS to be used over the entire volume of the tumor, if desired. This can result in more arbitrary treatment strategies, which may be helpful in the development of rapid ablation strategies, or strategies designed to minimize off target cavitation, such as cavitation on the surface of the skin.

It is important to note that the proposed design utilizes a composite (DL-53) piezoelectric. Use of composite, as opposed to a monolithic piezoelectric, such as PZ36 will affect performance in several ways. First, it can result in increased pressure output. This increase in performance will increase the likelihood of success with some aggressive treatment strategies such as the large focus strategy. Additionally, as the individual elements are only 7.4 mm wide, use of a composite material will help ensure uniform surface excitation.

Use of a composite piezoelectric also typically imposes a lower PRF limitation on the transducer. As a main objective of this transducer is to rapidly ablate large tumors, this is important, as it could limit treatment speed. The ability to subaperture the array and provide treatment to multiple distinct regions of the tumor, however, will aid in overcoming this limitation, as the global treatment PRF can remain high, while minimizing the PRF of individual subapertures or elements of the array.

Another significant benefit of the cylindrical design is ease of construction. As the array is only curved in one dimension, alternative strategies may be used. This fabrication method can significantly decrease the fabrication burden (time, financial cost, etc.) of the array in comparison to other histotripsy array fabrication techniques, while maintaining the benefits of modular construction.

Three main treatment strategies were proposed for the treatment of STS. The first, traditional EFS, has been shown to have potential as a high-rate ablation method. Multi-focal EFS, an extension of traditional EFS, was proposed for this particular indication, in part due to the cylindrical geometry of the array. Due to this geometry, when using traditional EFS, a minority of array elements are responsible for the majority of the focal pressure. This means the array can be efficiently sub-apertured to allow for multi-focal treatment. A spherically focused array with sufficient headroom, such as the one described above, could be used for multi-focal treatment, however, the driving voltage of the array would need to be increased approximately linearly with the number of subapertures used. This is because each element in the spherically focused array contributes an approximately equal amount to the focal pressure. In comparison, the cylindrical array would only require a minor increase in driving voltage to account for the sub-aperturization used for multi-focal treatment.

A novel, large focus treatment method is proposed, which results in a long, rod-shaped focal region exceeding the cavitation threshold. This focal region is much larger than a traditional EFS focal region, which may result in a much larger treatment region with a single pulse. This large focus cannot be electronically steered over a large region, so the transducer would need to be mechanically scanned over a volume to perform treatment. It is important to note that the length of this large focus can be adjusted by firing fewer elements on the periphery of the array, allowing the large focus technique to be used in tumors which are not as long as the array. For practical, rapid clinical treatment of STS, it is likely that a combination of these treatment strategies could be used for maximum benefit. For example, the large focus technique could be used to treat the central region of the tumor very rapidly, and the multi-focal EFS technique, which would allow for greater treatment precision, could be used to treat near the margins of the tumor.

While rapid ablation techniques are undoubtedly important for treating large STS, it will also be important to implement techniques for minimizing skin damage. As STS can be located very close to the skin, surface cavitation could become prominent, especially when treating near the periphery of the tumor volume. One approach for minimizing surface cavitation could involve turning off elements that are not essential to generate cavitation, and minimizing the driving voltage of others to lessen the pressure generated at the skin surface. Additionally, it will be necessary to ensure the coupling fluid used the water bath is highly degassed.

The novel, semi-cylindrical array design also offers potential to perform a variety of experiments to aid in developing a better fundamental understanding of the histotripsy mechanism. Spherically focused arrays typically can only generate histotripsy focal clouds with a maximum dimension on the order of a few wavelengths. The large focus treatment strategy will allow for new experiments to be designed to investigate cavitation behavior with larger bubble clouds. Commonly, histotripsy bubble clouds expand as a dense cloud of many bubbles, and violently collapse to a single point. Previous work has suggested that the collapse is primarily responsible for dam-aging the tissue. Due to the unusual size of the large focus, the collapse behavior of such a bubble cloud is of particular interest. If such a cloud collapses to a single point, it is feasible that the most strongly damaged area of tissue will be confined to the region immediately surrounding the particular point of collapse. Rather, if the cloud collapses to multiple points, damage could be distributed more evenly across the cloud volume. Experimentally, to answer this question of damage effectiveness with this treatment method, high speed photography will need to be used to determine the collapse process in the free-field (i.e. single or multiple collapse points) of the large cloud. After examining the free-field collapse process, progressive ablation of a red blood cell (RBC) phantom would allow damage extent and uniformity to be determined. Additionally, the bubble cloud formed using this treatment method differs from a cloud formed using a more typical histotripsy transducer geometry in that the long axis of the cloud is not aligned with the acoustic axis of the transducer. While this most likely will not affect the histotripsy damage mechanism itself, it may affect other cavitation cloud characteristics such as bubble cloud density or propensity for pre-focal shift.

### Electronic Steering

When using mechanical scanning over a treatment volume with histotripsy, a single location is typically repeatedly exposed to cavitation prior to progressing through the volume. When a histotripsy bubble cloud is formed, many cavitation bubbles rapidly expand and collapse. Time to collapse typically is on the order of a couple hundred microseconds, however, residual bubbles persist after collapse for much longer, typically in the range of 100-200 ms or longer. When using PRF greater than ≈5 Hz (corresponding to the time for residual bubbles to dissolve), it has been shown that these residual bubbles can act to re-excite cavitation, resulting in nearly identical bubble clouds being formed after the initial therapy pulse, with individual bubbles within the cloud forming at locations closely corresponding to individual bubbles of previous clouds. This can decrease the damage efficiency on a per pulse basis. This is known as the cavitation memory effect. Multiple studies have been published investigating the cavitation memory effect in histotripsy, as well as strategies to mitigate this effect. To mitigate this effect, both passive and active strategies have been employed. Passive mitigation of the memory effect consists of decreasing the PRF, and therefore increasing the pulse repetition period, such that remnant bubbles are allowed to passively dissolve prior to subsequent therapy pulses being applied. Utilization of this passive approach is problematic during large volume treatments, as treatment times increase to unacceptable levels for clinical use. Alternatively, active bubble coalescence strategies have been proposed and implemented, which consist of multiple sub-threshold acoustic pulses being applied between therapy pulses. While this strategy has been shown to be effective in lessening the memory effect, the additional coalescence pulsing can increase heating of overlying tissue, which can impose an effective treatment rate limit.

To avoid the additional pulsing associated with active bubble coalescence strategies, an alternative has been proposed. By using a phased array transducer, the focal location can be electronically steered to arbitrary points throughout the treatment volume. By designing the steering location sequence to avoid previously treated locations, an entire volume may be treated with a high global PRF, while maintaining low local PRFs at each individual location or region. Additionally, when using EFS, low gain regions of the pressure field can serve to promote bubble coalescence of residual bubbles. Previously, these techniques have been used with a high degree of success when implemented with a low frequency (250 kHz) hemispherical histotripsy transducer.

It should be noted that much of the work performed showing the cavitation memory effect and developing mitigation strategies (both active and passive) has relied strongly on use of transparent agarose-based phantoms to facilitate the use high speed photography to visualize individual cavitation clouds. This approach assumes cavitation processes (expansion, collapse, dissolution, re-excitation, etc.) are identical in agarose and biologic tissue. Unfortunately, the level of correlation between these processes in agarose and tissue is unclear. Additionally, histotripsy instrumentation has progressed significantly since many of these studies were performed, and current generation systems are often higher power and higher bandwidth than previous systems. These distinctions may play a significant role in the damage efficiency of different histotripsy treatment strategies.

A goal of this disclosure is to expand upon the previous histotripsy EFS work by further exploring the treatment parameter space and EFS sequencing, as well as implementing these techniques with a higher frequency (750 kHz), non-hemispherical, current generation transducer designed for targeting the human abdominal region. The combined effects of focal pressure, dose (pulses per cubic centimeter), and PRF are first examined for a given transducer power output and treatment time in ex-vivo bovine liver tissue. Next, the effect of EFS point spacing is explored. Using optimized treatment parameters, several EFS sequencing strategies are proposed, implemented, and tested.

Previously, it has been shown that histotripsy bubble clouds generated with higher peak negative pressures are larger overall, and also feature larger individual bubbles and higher bubble density. This was shown to result in higher damage efficiency in red blood cell phantoms on a per pulse basis. The question remains whether higher pressure results in higher damage efficiency in tissue if the same acoustic power and energy is used. To explore this, the following experiment was performed in ex-vivo bovine liver tissue. The array was programmed to ablate 2 cm diameter spherical volumes using EFS and a range of focal pressures (28-80 MPa). Volumes were con-structed from 5,918 points, hexagonally close packed with a spacing of 1 mm. Driving voltage for each EFS location was calculated to account for the steerability of the array, resulting in uniform pressure being applied to each EFS location. The EFS location coordinates were identical for all ablations. The dose was set based on previous experience to result in partial ablation. In conjunction with focal pressure, the dose and PRF were varied between ablations to maintain a constant input power.

Previous work on histotripsy EFS techniques for rapid ablation have typically relied on low frequency (250 kHz) transducers and relatively coarse EFS grid spacings (2.5+ mm). The decision to use coarse spacing was made in part due to system limitations on the quantity of focal locations the system was capable of storing, and was enabled by the large cavitation clouds generated by the relatively low frequency. To fully ablate tissue using this EFS strategy, the array must be fired at each EFS location many times (>100). Due to system advances, it is now feasible to define large volume EFS grids with many more focal points, with much finer spacing (<1 mm). It was hypothesized that by decreasing the spacing of EFS points, and therefore increasing the EFS point density, a more homogeneous lesion could be ablated in with a lower dose. To test this hypothesis, a series of ablations at 3 spacings (0.5, 1, and 2 mm) was performed. For reference, the −6 dB focal volume of the transducer is 1.6×1.1×4.5 mm. The number of repetitions at each location was varied to account for the difference in point density, resulting in the same dose being applied for each spacing. EFS locations were ordered randomly, and a single pulse was fired at each point before returning for subsequent repetitions. All ablations were performed with a PRF of 1000 Hz, and a focal pressure of 40 MPa. The same structured EFS location sequence as for the pressure optimization ablations was used for the spacing optimization ablations.

To evaluate the performance of different EFS sequences, ablations were performed using four distinct sequences at three dose (acoustic pulses applied per cc treatment volume) levels (12 different dose-sequence combinations). The first sequence was a simple raster scan, which was included to determine what effect the cavitation memory effect has on ablation efficiency, in comparison with sequencing strategies designed to lessen its effect. The second sequence was a structured sequence designed to lessen the local PRFs within small regions of the ablation volume. A third sequence (Anti-Shielding) was designed to minimize acoustic energy shielding of the intended focus. Acoustic shielding from remnant bubbles can occur when residual bubbles from a previous pulse remain in the acoustic path from the transducer to the current intended focal location. Remnant pre-focal bubbles can block sound from reaching the subsequent intended location, decreasing the likelihood of a robust cavitation cloud forming at the intended focus (especially when using high PRFs with limited time for dissolution prior to subsequent pulses). The last sequence (Interface) was designed to take advantage of the lowered cavitation threshold on tissue interfaces. By ablating tissue completely on the side of the ablation volume nearest the transducer, and then progressing through the volume, an interface is maintained at the intended ablation location. All volumes ablated for the dose-sequence ablations were 2 cm diameter spherical volumes, consisting of 5,918 hexagonally close packed points, with point spacing of 1 mm. Ablations were performed with a normalized focal pressure of 56.6 MPa, and a PRF of 500 Hz. Tissue was prepared identically to previous ex-vivo ablations.

### Raster EFS Sequence

Points were ordered using a standard raster pattern, starting with points in the layer furthest (distal) from the transducer and moving in the towards the transducer (proximal). The distal to proximal ordering was done to minimize the likelihood of pre-focal remnant bubbles shielding sound from the intended focal location. The transducer was fired at each point N times before progressing through the sequence to the next (adjacent) point. One repetition was made through the sequence. Note that this sequence is the only sequence designed to repeatedly expose a single location to multiple therapy pulses prior to progressing to the next EFS location, similar to mechanically scanning the transducer along the prescribed path.

### Structured EFS Sequence

This sequence is similar to the sequence proposed above to reduce cavitation memory effect, with slight modifications. Points were divided into sub-grids which ensured a minimum spacing of 5 mm in the lateral directions between all pairs of points within each sub-grid. Within sub-grids, points were ordered distal to proximal along the acoustic axis, with a random sequence of points at the same axial distance from the transducer. Each point was fired at once before moving on to subsequent points within the sub-grid. Once each sub-grid was completed, the process was completed with the next sub-grid, until every sub-grid had been treated (one pulse applied at each individual location). This entire process was repeated N times to complete the treatment.

### Anti-Shielding EFS Sequence

Using the size and shape of the transducer aperture, points were divided into layered groups which serve to order the points such that acoustic shielding is guaranteed to not occur. This results in a first group that is a spherical shell shaped layer located at the most distal boundary of the intended volume, and subsequent layers which become progressively flatter as they progress closer to the proximal boundary of the volume. Starting with the first (most distal) layer, points were ordered randomly, and a single treatment pulse was applied to each point in the layer. N repetitions were completed on each layer before progressing to the next layer. This lessened the probability of any pre-focal bubbles being formed, thus decreasing the likelihood that the shielding effect would decrease treatment efficacy.

### Interface EFS Sequence

The interface sequence was identical to the anti-shielding sequence, but was performed in reverse (proximal to distal). It is hypothesized that by fully ablating the proximal locations within the volume first, an interface between liquefied and un-ablated tissue could be formed, which may aid in forming cavitation clouds at the liquefied tissue interface and efficiently ablating tissue.

One area of concern when attempting large volume, rapid ablation with histotripsy is the potential for off-target damage due to heat deposition. As focal pressure and PRF are increased in an attempt to increase ablation rate, more energy is deposited into overlying tissues, increasing the potential for off-target, unintended injury. Further studies will need to be performed to establish the thermal safety of the pulsing scheme presented here. While the study presented in this paper does not explicitly explore the potential for such damage, the methodology was designed such that results from the optimization should hold for adjustments to PRF. Because the focal pressure optimization held acoustic power constant for all focal pressures, the approximate heat deposition should be similar for each parameter set tested. It should be noted that the range of duty cycle used in this study is low (0.05%-0.4%), which is expected to mitigate the thermal effect. In the case that acoustic power needs to be decreased in order to ensure patient safety with the described pulsing scheme, two options are present. One option would be to decrease PRF to lower the acoustic input to a safe level. Alternatively, intermittent cooling pauses of treatment could be implemented to allow tissue to allow tissue to cool prior to continuing treatment.

An observation of particular interest was the ablation efficiency dependence on focal pressure. Even with a reduced number of repetitions over the treatment volume, substantially more damage was done with a focal pressure of 56.6 MPa, as compared with lower focal pressures. This is likely due to a number of different factors. Previously, it has been shown that histotripsy bubble clouds formed with higher peak negative pressure show increased bubble quantity and density. Histotripsy bubble clouds are also typically larger when generated with higher pressures, as a larger focal region will exceed the intrinsic threshold. We believe that the increased bubble quantity, density, and cloud size substantially increase the damage sustained by each pulse when increasing the focal pressure over the range of 40-56 MPa. The effect seems to reach a saturation prior to 80 MPa, when overall damage is decreased, likely due to the decreased repetitions applied at the higher pressure.

Because relatively high focal pressure showed the highest damage efficiency, there is concern about translating the presented ablation techniques to in-vivo scenarios with limited acoustic windows. Specifically for treatment of the liver, some regions may require transcostal treatment, which may limit focal pressure amplitude, even if effective aberration correction algorithms are used. To optimize pulsing strategy for the most rapid ablation in these cases, an adaptive ablation strategy may need to be employed, using higher repetition (lower focal pressure) treatment in blocked regions, and higher pressure, more rapid treatment in regions of the liver with better acoustic access. Such a strategy would likely need to be developed in combination with advanced targeting techniques, such as X-ray C-arm guided targeting techniques, which could be used to predict what target regions would involve partially blocked acoustic windows. Alternatively, shock-scattering histotripsy could be used in obstructed regions to lower the cavitation initiation threshold while maintaining dense bubble cloud formation.

No significant effect of EFS spacing on damage efficiency was detected over the range of 0.5-2 mm focal spacing. This is likely due to the focal volume of the transducer, which measures 1.6×1.1×4.5 mm. While the lateral dimensions of the FWHM are less than the 2 mm spacing, substantial overlap from axially adjacent focal locations likely renders this range of spacings within the acceptable range for achieving homogenous damage. It should be noted that the acceptable focal spacing range will be heavily affected by the transducer used (aperture, frequency, etc.). For transducers featuring a higher operating frequency or lower f #, it is likely that the acceptable range of focal spacings will be lower than for lower frequency or higher f #transducers.

With the experimental setup and treatment parameters used in this experiment, >95% of cells within the treatment volume were homogenized within approximately 11,000 pulses/cc of treatment for both layered EFS sequences, resulting in a treatment rate of 2.65 cc/min. For the purposes of this experiment, complete treatment (>95% of cells) was chosen as the target treatment end point, however, it is still unclear what level of treatment is optimal for many indications. Currently, partial treatment of tumors with histotripsy is an active area of research. Studies have shown potential for partial treatments to stimulate an immune response, resulting in local and abscopal responses to treatment. Thus, effective clinical treatment rate may be affected by further research into the optimal treatment endpoint.

While the results of this study indicated that a focal pressure of 56 MPa was the most effective of those tested, it is possible that further optimization of different EFS sequences could enable similarly rapid ablation with different parameters. For example, a raster-based strategy could enable effective treatment with lower focal pressures and a high PRF, as it would take advantage of a lowered cavitation threshold due to residual bubbles persisting at the intended focal location. Additionally, this strategy would benefit from the shock scattering mechanism, which takes advantage of high peak positive pressures being reflected off of existing bubble clouds to maintain a robust cavitation cloud.

An interesting finding of the dose-sequence ablation comparison was the relative lack of difference between the required dose for different sequences. Previous studies have shown a high dependence on local PRF and the required number of pulses to fully homogenize tissue. The raster (high local PRF) and structured (low local PRF) sequence required approximately the same dose to fully treat tissue in this experiment, which seems to contradict previous results. First, the electrical driving system in this experiment is of much higher bandwidth compared to the previous study. The driver used in the current study outputs a clean 1-cycle sine wave, while the previously used driver output a 10-cycle tone burst driving pulse. This may greatly affect the observed memory effect, as the highest amplitude peak negative pressure occurs on ap-proximately the 10th acoustic peak negative, meaning that lower (sub-threshold) pulses repeatedly excite the residual nuclei immediately prior to the cavitation-inducing pulse. The current system reaches its maximum peak negative on the first negative portion of the acoustic pulse. This may allow new cavitation clouds to be less correlated with previous clouds. Additionally, the peak negative focal pressure used may also impact the degree to which the memory effect is observed. The intrinsic threshold of cavitation in liver tissue has been measured to be in the range of 17-20 MPa for PRFs in the range of 100-1000 Hz. The previous study used a focal pressure just above the threshold (21 MPa), while the present study used a pressure nearly 3× the threshold (56 MPa). By increasing the focal pressure to this level, it may be possible to mitigate the memory effect almost entirely, resulting in high damage efficiency on a per-pulse basis.

In addition to the large focus treatment strategy, multi-focal treatment strategies also are also contemplated using the STS array. Due to the cylindrical shape of the array, a unique opportunity to employ efficient subaperturization exists without substantially increasing the input acoustic energy (and associated heating and strain on the transducer) necessary for treatment.

Because STS generally are located very near the skin surface, precautions will need to be taken to minimize the likelihood of surface cavitation causing significant damage to the skin. One potential method of minimizing surface cavitation is decreasing the focal pressure when treating portions of the treatment volume near the skin surface. This will likely require a higher dose (pulses/cc) to achieve complete ablation. In turn, this will decrease ablation rate, but should result in lessened surface cavitation and skin damage. Alternatively, coupling fluids other than degassed water could be explored. By using a coupling fluid with a higher intrinsic threshold of cavitation, surface cavitation may be decreased, allowing for higher focal pressures (and associated ablation rates) to be deemed safe for regions near the skin surface.

In the event that multiple treatment strategies (large focus, multi-focal, traditional EFS), are successful, it may be beneficial to devise a combination strategy to utilize the strength of each. For example, first, the large focus strategy could be used to very rapidly ablate the central region of the tumor. Then, multi-focal treatment could be used to more precisely target the edges of the tumor, or regions outside of the targetable large-focus zone. To realistically apply this in an in-vivo setting, an algorithm would need to be written to quickly segment regions of the tumor for each treatment strategy.

### Integrated Imaging

The disclosed system may comprise various imaging modalities to allow users to visualize, monitor and collect/use feedback of the patient's anatomy, related regions of interest and treatment/procedure sites, as well as surrounding and intervening tissues to assess, plan and conduct procedures, and adjust treatment parameters as needed. Imaging modalities may comprise various ultrasound, x-ray, CT, MRI, PET, fluoroscopy, optical, contrast or agent enhanced versions, and/or various combinations of. It is further disclosed that various image processing and characterization technologies may also be utilized to afford enhanced visualization and user decision making. These may be selected or commanded manually by the user or in an automated fashion by the system. The system may be configured to allow side by side, toggling, overlays, 3D reconstruction, segmentation, registration, multi-modal image fusion, image flow, and/or any methodology affording the user to identify, define and inform various aspects of using imaging during the procedure, as displayed in the various system user interfaces and displays. Examples may include locating, displaying and characterizing regions of interest, organ systems, potential treatment sites within, with on and/or surrounding organs or tissues, identifying critical structures such as ducts, vessels, nerves, ureters, fissures, capsules, tumors, tissue trauma/injury/disease, other organs, connective tissues, etc., and/or in context to one another, of one or more (e.g., tumor draining lymphatics or vasculature; or tumor proximity to organ capsule or underlying other organ), as unlimited examples.

Systems may be configured to include onboard integrated imaging hardware, software, sensors, probes and wetware, and/or may be configured to communicate and interface with external imaging and image processing systems. The aforementioned components may be also integrated into the system's Therapy sub-system components wherein probes, imaging arrays, or the like, and electrically, mechanically or electromechanically integrated into therapy transducers. This may afford, in part, the ability to have geometrically aligned imaging and therapy, with the therapy directly within the field of view, and in some cases in line, with imaging. In some embodiments, this integration may comprise a fixed orientation of the imaging capability (e.g., imaging probe) in context to the therapy transducer. In other embodiments, the imaging solution may be able to move or adjust its position, including modifying angle, extension (e.g., distance from therapy transducer or patient), rotation (e.g., imaging plane in example of an ultrasound probe) and/or other parameters, including moving/adjusting dynamically while actively imaging. The imaging component or probe may be encoded so its orientation and position relative to another aspect of the system, such as the therapy transducer, and/or robotically-enabled positioning component may be determined.

In one embodiment, the system may comprise onboard ultrasound, further configured to allow users to visualize, monitor and receive feedback for procedure sites through the system displays and software, including allowing ultrasound imaging and characterization (and various forms of), ultrasound guided planning and ultrasound guided treatment, all in real-time. The system may be configured to allow users to manually, semi-automated or in fully automated means image the patient (e.g., by hand or using a robotically-enabled imager).

In some embodiments, imaging feedback and monitoring can include monitoring changes in: backscatter from bubble clouds; speckle reduction in backscatter; backscatter speckle statistics; mechanical properties of tissue (i.e., elastography); tissue perfusion (i.e., ultrasound contrast); shear wave propagation; acoustic emissions, electrical impedance tomography, and/or various combinations of, including as displayed or integrated with other forms of imaging (e.g., CT or MRI).

In some embodiments, imaging including feedback and monitoring from backscatter from bubble clouds, may be used as a method to determine immediately if the histotripsy process has been initiated, is being properly maintained, or even if it has been extinguished. For example, this method enables continuously monitored in real time drug delivery, tissue erosion, and the like. The method also can provide feedback permitting the histotripsy process to be initiated at a higher intensity and maintained at a much lower intensity. For example, backscatter feedback can be monitored by any transducer or ultrasonic imager. By measuring feedback for the therapy transducer, an accessory transducer can send out interrogation pulses or be configured to passively detect cavitation. Moreover, the nature of the feedback received can be used to adjust acoustic parameters (and associated system parameters) to optimize the drug delivery and/or tissue erosion process.

In some embodiments, imaging including feedback and monitoring from backscatter, and speckle reduction, may be configured in the system.

For systems comprising feedback and monitoring via backscattering, and as means of background, as tissue is progressively mechanically subdivided, in other words homogenized, disrupted, or eroded tissue, this process results in changes in the size and distribution of acoustic scatter. At some point in the process, the scattering particle size and density is reduced to levels where little ultrasound is scattered, or the amount scattered is reduced significantly. This results in a significant reduction in speckle, which is the coherent constructive and destructive interference patterns of light and dark spots seen on images when coherent sources of illumination are used; in this case, ultrasound. After some treatment time, the speckle reduction results in a dark area in the therapy volume. Since the amount of speckle reduction is related to the amount of tissue subdivision, it can be related to the size of the remaining tissue fragments. When this size is reduced to sub-cellular levels, no cells are assumed to have survived. So, treatment can proceed until a desired speckle reduction level has been reached. Speckle is easily seen and evaluated on standard ultrasound imaging systems. Specialized transducers and systems, including those disclosed herein, may also be used to evaluate the backscatter changes.

Further, systems comprising feedback and monitoring via speckle, and as means of background, an image may persist from frame to frame and change very little as long as the scatter distribution does not change and there is no movement of the imaged object. However, long before the scatters are reduced enough in size to cause speckle reduction, they may be changed sufficiently to be detected by signal processing and other means. This family of techniques can operate as detectors of speckle statistics changes. For example, the size and position of one or more speckles in an image will begin to decorrelate before observable speckle reduction occurs. Speckle decorrelation, after appropriate motion compensation, can be a sensitive measure of the mechanical disruption of the tissues, and thus a measure of therapeutic efficacy. This feedback and monitoring technique may permit early observation of changes resulting from the acoustic cavitation/histotripsy process and can identify changes in tissue before substantial or complete tissue effect (e.g., erosion occurs). In one embodiment, this method may be used to monitor the acoustic cavitation/histotripsy process for enhanced drug delivery where treatment sites/tissue is temporally disrupted, and tissue damage/erosion is not desired. In other embodiments, this may comprise speckle decorrelation by movement of scatters in an increasingly fluidized therapy volume. For example, in the case where partial or complete tissue erosion is desired.

For systems comprising feedback and monitoring via elastography, and as means of background, as treatment sites/tissue are further subdivided per an acoustic cavitation/histotripsy effect (homogenized, disrupted, or eroded), its mechanical properties change from a soft but interconnected solid to a viscous fluid or paste with few long-range interactions. These changes in mechanical properties can be measured by various imaging modalities including MRI and ultrasound imaging systems. For example, an ultrasound pulse can be used to produce a force (i.e., a radiation force) on a localized volume of tissue. The tissue response (displacements, strains, and velocities) can change significantly during histotripsy treatment allowing the state of tissue disruption to be determined by imaging or other quantitative means.

Systems may also comprise feedback and monitoring via shear wave propagation changes. As means of background, the subdivision of tissues makes the tissue more fluid and less solid and fluid systems generally do not propagate shear waves. Thus, the extent of tissue fluidization provides opportunities for feedback and monitoring of the histotripsy process. For example, ultrasound and MRI imaging systems can be used to observe the propagation of shear waves. The extinction of such waves in a treated volume is used as a measure of tissue destruction or disruption. In one system embodiment, the system and supporting sub-systems may be used to generate and measure the interacting shear waves. For example, two adjacent ultrasound foci might perturb tissue by pushing it in certain ways. If adjacent foci are in a fluid, no shear waves propagate to interact with each other. If the tissue is not fluidized, the interaction would be detected with external means, for example, by a difference frequency only detected when two shear waves interact nonlinearly, with their disappearance correlated to tissue damage. As such, the system may be configured to use this modality to enhance feedback and monitoring of the acoustic cavitation/histotripsy procedure.

For systems comprising feedback and monitoring via acoustic emission, and as means of background, as a tissue volume is subdivided, its effect on acoustic cavitation/histotripsy (e.g., the bubble cloud here) is changed. For example, bubbles may grow larger and have a different lifetime and collapse changing characteristics in intact versus fluidized tissue. Bubbles may also move and interact after tissue is subdivided producing larger bubbles or cooperative interaction among bubbles, all of which can result in changes in acoustic emission. These emissions can be heard during treatment, and they change during treatment. Analysis of these changes, and their correlation to therapeutic efficacy, enables monitoring of the progress of therapy, and may be configured as a feature of the system.

For systems comprising feedback and monitoring via electrical impedance tomography, and as means of background, an impedance map of a therapy site can be produced based upon the spatial electrical characteristics throughout the therapy site. Imaging of the conductivity or permittivity of the therapy site of a patient can be inferred from taking skin surface electrical measurements. Conducting electrodes are attached to a patient's skin and small alternating currents are applied to some or all of the electrodes. One or more known currents are injected into the surface and the voltage is measured at a number of points using the electrodes. The process can be repeated for different configurations of applied current. The resolution of the resultant image can be adjusted by changing the number of electrodes employed. A measure of the electrical properties of the therapy site within the skin surface can be obtained from the impedance map, and changes in and location of the acoustic cavitation/histotripsy (e.g., bubble cloud, specifically) and histotripsy process can be monitored using this as configured in the system and supporting sub-systems.

The user may be allowed to further select, annotate, mark, highlight, and/or contour, various regions of interest or treatment sites, and defined treatment targets (on the image(s)), of which may be used to command and direct the system where to image, test and/or treat, through the system software and user interfaces and displays. In some arrangements, the user may use a manual ultrasound probe (e.g., diagnostic hand-held probe) to conduct the procedure. In another arrangement, the system may use a robot and/or electromechanical positioning system to conduct the procedure, as directed and/or automated by the system, or conversely, the system can enable combinations of manual and automated uses.

The system may further include the ability to conduct image registration, including imaging and image data set registration to allow navigation and localization of the system to the patient, including the treatment site (e.g., tumor, critical structure, bony anatomy, anatomy and identifying features of, etc.). In one embodiment, the system allows the user to image and identify a region of interest, for example the liver, using integrated ultrasound, and to select and mark a tumor (or surrogate marker of) comprised within the liver through/displayed in the system software, and wherein said system registers the image data to a coordinate system defined by the system, that further allows the system's Therapy and Robotics sub-systems to deliver synchronized acoustic cavitation/histotripsy to said marked tumor. The system may comprise the ability to register various image sets, including those previously disclosed, to one another, as well as to afford navigation and localization (e.g., of a therapy transducer to a CT or MRI/ultrasound fusion image with the therapy transducer and Robotics sub-system tracking to said image).

The system may also comprise the ability to work in a variety of interventional, endoscopic and surgical environments, including alone and with other systems (surgical/laparoscopic towers, vision systems, endoscope systems and towers, ultrasound enabled endoscopic ultrasound (flexible and rigid), percutaneous/endoscopic/laparoscopic and minimally invasive navigation systems (e.g., optical, electromagnetic, shape-sensing, ultrasound-enabled, etc.), of also which may work with, or comprise various optical imaging capabilities (e.g., fiber and or digital). The disclosed system may be configured to work with these systems, in some embodiments working alongside them in concert, or in other embodiments where all or some of the system may be integrated into the above systems/platforms (e.g., acoustic cavitation/histotripsy-enabled endoscope system or laparoscopic surgical robot). In many of these environments, a therapy transducer may be utilized at or around the time of use, for example, of an optically guided endoscope/bronchoscope, or as another example, at the time a laparoscopic robot (e.g., Intuitive Da Vinci* Xi system) is viewing/manipulating a tissue/treatment site. Further, these embodiments and examples may include where said other systems/platforms are used to deliver (locally) fluid to enable the creation of a man-made acoustic window, where on under normal circumstances may not exist (e.g., fluidizing a segment or lobe of the lung in preparation for acoustic cavitation/histotripsy via non-invasive transthoracic treatment (e.g., transducer externally placed on/around patient). Systems disclosed herein may also comprise all or some of their sub-system hardware packaged within the other system cart/console/systems described here (e.g., acoustic cavitation/histotripsy system and/or sub-systems integrated and operated from said navigation or laparoscopic system).

The system may also be configured, through various aforementioned parameters and other parameters, to display real-time visualization of a bubble cloud in a spatial-temporal manner, including the resulting tissue effect peri/post-treatment from tissue/bubble cloud interaction, wherein the system can dynamically image and visualize, and display, the bubble cloud, and any changes to it (e.g., decreasing or increasing echogenicity), which may include intensity, shape, size, location, morphology, persistence, etc. These features may allow users to continuously track and follow the treatment in real-time in one integrated procedure and interface/system, and confirm treatment safety and efficacy on the fly (versus other interventional or surgical modalities, which either require multiple procedures to achieve the same, or where the treatment effect is not visible in real-time (e.g., radiation therapy), or where it is not possible to achieve such (e.g., real-time visualization of local tissue during thermal ablation), and/or where the other procedure further require invasive approaches (e.g., incisions or punctures) and iterative imaging in a scanner between procedure steps (e.g., CT or MRI scanning). The above disclosed systems, sub-systems, components, modalities, features and work-flows/methods of use may be implemented in an unlimited fashion through enabling hardware, software, user interfaces and use environments, and future improvements, enhancements and inventions in this area are considered as included in the scope of this disclosure, as well as any of the resulting data and means of using said data for analytics, artificial intelligence or digital health applications and systems.

### Robotics

They system may comprise various Robotic sub-systems and components, including but not limited to, one or more robotic arms and controllers, which may further work with other sub-systems or components of the system to deliver and monitor acoustic cavitation/histotripsy. As previously discussed herein, robotic arms and control systems may be integrated into one or more Cart configurations.

For example, one system embodiment may comprise a Cart with an integrated robotic arm and control system, and Therapy, Integrated Imaging and Software, where the robotic arm and other listed sub-systems are controlled by the user through the form factor of a single bedside Cart.

In other embodiments, the Robotic sub-system may be configured in one or more separate Carts, that may be a driven in a master/slave configuration from a separate master or Cart, wherein the robotically-enabled Cart is positioned bed/patient-side, and the Master is at a distance from said Cart.

Disclosed robotic arms may be comprised of a plurality of joints, segments, and degrees of freedom and may also include various integrated sensor types and encoders, implemented for various use and safety features. Sensing technologies and data may comprise, as an example, vision, potentiometers, position/localization, kinematics, force, torque, speed, acceleration, dynamic loading, and/or others. In some cases, sensors may be used for users to direct robot commands (e.g., hand gesture the robot into a preferred set up position, or to dock home). Additional details on robotic arms can be found in US Patent Pub. No. 2013/0255426 to Kassow et al. which is disclosed herein by reference in its entirety.

The robotic arm receives control signals and commands from the robotic control system, which may be housed in a Cart. The system may be configured to provide various functionalities, including but not limited to, position, tracking, patterns, triggering, and events/actions.

Position may be configured to comprise fixed positions, pallet positions, time-controlled positions, distance-controlled positions, variable-time controlled positions, variable-distance controlled positions.

Tracking may be configured to comprise time-controlled tracking and/or distance-controlled tracking.

The patterns of movement may be configured to comprise intermediate positions or waypoints, as well as sequence of positions, through a defined path in space.

Triggers may be configured to comprise distance measuring means, time, and/or various sensor means including those disclosed herein, and not limited to, visual/imaging-based, force, torque, localization, energy/power feedback and/or others.

Events/actions may be configured to comprise various examples, including proximity-based (approaching/departing a target object), activation or de-activation of various end-effectors (e.g., therapy transducers), starting/stopping/pausing sequences of said events, triggering or switching between triggers of events/actions, initiating patterns of movement and changing/toggling between patterns of movement, and/or time-based and temporal over the defined work and time-space.

In one embodiment, the system comprises a three degree of freedom robotic positioning system, enabled to allow the user (through the software of the system and related user interfaces), to micro-position a therapy transducer through X, Y, and Z coordinate system, and where gross macro-positioning of the transducer (e.g., aligning the transducer on the patient's body) is completed manually. In some embodiments, the robot may comprise 6 degrees of freedom including X, Y, Z, and pitch, roll and yaw. In other embodiments, the Robotic sub-system may comprise further degrees of freedom, that allow the robot arm supporting base to be positioned along a linear axis running parallel to the general direction of the patient surface, and/or the supporting base height to be adjusted up or down, allowing the position of the robotic arm to be modified relative to the patient, patient surface, Cart, Coupling sub-system, additional robots/robotic arms and/or additional surgical systems, including but not limited to, surgical towers, imaging systems, endoscopic/laparoscopic systems, and/or other.

One or more robotic arms may also comprise various features to assist in maneuvering and modifying the arm position, manually or semi-manually, and of which said features may interface on or between the therapy transducer and the most distal joint of the robotic arm. In some embodiments, the feature is configured to comprise a handle allowing maneuvering and manual control with one or more hands. The handle may also be configured to include user input and electronic control features of the robotic arm, to command various drive capabilities or modes, to actuate the robot to assist in gross or fine positioning of the arm (e.g., activating or deactivating free drive mode). The work-flow for the initial positioning of the robotic arm and therapy head can be configured to allow either first positioning the therapy transducer/head in the coupling solution, with the therapy transducer directly interfaced to the arm, or in a different work-flow, allowing the user to set up the coupling solution first, and enabling the robot arm to be interfaced to the therapy transducer/coupling solution as a later/terminal set up step.

In some embodiments, the one or more robotic arms or other features of the robotic sub-systems may include sensors or other features configured to measure, determine, or predict the force(s) acting against the robotic arm(s) and/or the therapy transducer array coupled to the robotic arm(s). These sensors can include force sensors or force transducers not limited to load cells, pneumatic load cells, capacitive load cells, strain gauge load cells, hydraulic load cells, etc. In some implementations, the force sensors can be disposed on or in the robotic arm(s), on or in the transducer array or therapy probe, on or in the coupling linkages between the transducer array and robotic arm, or in any other location within the system, including the robotics sub-system, where a force sensor or sensors would be adapted and configured to measure the force applied against the robotic arm or the transducer array.

Additionally, these force sensors can be electronically or operatively coupled to any of the control systems described herein, including electronic controllers, robotic positioning systems, navigation systems, or any other cpus, processors, or controllers configured to control the operation of the transducer array, robotics sub-system, or any other sub-system during therapy.

In some embodiments, the robotic arm may comprise a robotic arm on a laparoscopic, single port, endoscopic, hybrid or combination of, and/or other robot, wherein said robot of the system may be a slave to a master that controls said arm, as well as potentially a plurality of other arms, equipped to concurrently execute other tasks (vision, imaging, grasping, cutting, ligating, sealing, closing, stapling, ablating, suturing, marking, etc.), including actuating one or more laparoscopic arms (and instruments) and various histotripsy system components. For example, a laparoscopic robot may be utilized to prepare the surgical site, including manipulating organ position to provide more ideal acoustic access and further stabilizing said organ in some cases to minimize respiratory motion. In conjunction and parallel to this, a second robotic arm may be used to deliver non-invasive acoustic cavitation through a body cavity, as observed under real-time imaging from the therapy transducer (e.g., ultrasound) and with concurrent visualization via a laparoscopic camera. In other related aspects, a similar approach may be utilized with a combination of an endoscopic and non-invasive approach, and further, with a combination of an endoscopic, laparoscopic and non-invasive approach.

### Software

The system may comprise various software applications, features and components which allow the user to interact, control and use the system for a plethora of clinical applications. The Software may communicate and work with one or more of the sub-systems, including but not limited to Therapy, Integrated Imaging, Robotics and Other Components, Ancillaries and Accessories of the system.

Overall, in no specific order of importance, the software may provide features and support to initialize and set up the system, service the system, communicate and import/export/store data, modify/manipulate/configure/control/command various settings and parameters by the user, mitigate safety and use-related risks, plan procedures, provide support to various configurations of transducers, robotic arms and drive systems, function generators and amplifier circuits/slaves, test and treatment ultrasound sequences, transducer steering and positioning (electromechanical and electronic beam steering, etc.), treatment patterns, support for imaging and imaging probes, manual and electromechanical/robotically-enabling movement of, imaging support for measuring/characterizing various dimensions within or around procedure and treatment sites (e.g., depth from one anatomical location to another, etc., pre-treatment assessments and protocols for measuring/characterizing in situ treatment site properties and conditions (e.g., acoustic cavitation/histotripsy thresholds and heterogeneity of), targeting and target alignment, calibration, marking/annotating, localizing/navigating, registering, guiding, providing and guiding through work-flows, procedure steps, executing treatment plans and protocols autonomously, autonomously and while under direct observation and viewing with real-time imaging as displayed through the software, including various views and viewports for viewing, communication tools (video, audio, sharing, etc.), troubleshooting, providing directions, warnings, alerts, and/or allowing communication through various networking devices and protocols. It is further envisioned that the software user interfaces and supporting displays may comprise various buttons, commands, icons, graphics, text, etc., that allow the user to interact with the system in a user-friendly and effective manner, and these may be presented in an unlimited number of permutations, layouts and designs, and displayed in similar or different manners or feature sets for systems that may comprise more than one display (e.g., touch screen monitor and touch pad), and/or may network to one or more external displays or systems (e.g., another robot, navigation system, system tower, console, monitor, touch display, mobile device, tablet, etc.).

The software, as a part of a representative system, including one or more computer processors, may support the various aforementioned function generators (e.g., FPGA), amplifiers, power supplies and therapy transducers. The software may be configured to allow users to select, determine and monitor various parameters and settings for acoustic cavitation/histotripsy, and upon observing/receiving feedback on performance and conditions, may allow the user to stop/start/modify said parameters and settings.

The software may be configured to allow users to select from a list or menu of multiple transducers and support the auto-detection of said transducers upon connection to the system (and verification of the appropriate sequence and parameter settings based on selected application). In other embodiments, the software may update the targeting and amplifier settings (e.g., channels) based on the specific transducer selection. The software may also provide transducer recommendations based on pre-treatment and planning inputs. Conversely, the software may provide error messages or warnings to the user if said therapy transducer, amplifier and/or function generator selections or parameters are erroneous, yield a fault or failure. This may further comprise reporting the details and location of such.

In addition to above, the software may be configured to allow users to select treatment sequences and protocols from a list or menu, and to store selected and/or previous selected sequences and protocols as associated with specific clinical uses or patient profiles. Related profiles may comprise any associated patient, procedure, clinical and/or engineering data, and maybe used to inform, modify and/or guide current or future treatments or procedures/interventions, whether as decision support or an active part of a procedure itself (e.g., using serial data sets to build and guide new treatments).

As a part of planning or during the treatment, the software (and in working with other components of the system) may allow the user to evaluate and test acoustic cavitation/histotripsy thresholds at various locations in a user-selected region of interest or defined treatment area/volume, to determine the minimum cavitation thresholds throughout said region or area/volume, to ensure treatment parameters are optimized to achieve, maintain and dynamically control acoustic cavitation/histotripsy. In one embodiment, the system allows a user to manually evaluate and test threshold parameters at various points. Said points may include those at defined boundary, interior to the boundary and center locations/positions, of the selected region of interest and treatment area/volume, and where resulting threshold measurements may be reported/displayed to the user, as well as utilized to update therapy parameters before treatment. In another embodiment, the system may be configured to allow automated threshold measurements and updates, as enabled by the aforementioned Robotics sub-system, wherein the user may direct the robot, or the robot may be commanded to execute the measurements autonomously.

Software may also be configured, by working with computer processors and one or more function generators, amplifiers and therapy transducers, to allow various permutations of delivering and positioning optimized acoustic cavitation/histotripsy in and through a selected area/volume. This may include, but not limited to, systems configured with a fixed/natural focus arrangement using purely electromechanical positioning configuration(s), electronic beam steering (with or without electromechanical positioning), electronic beam steering to a new selected fixed focus with further electromechanical positioning, axial (Z axis) electronic beam steering with lateral (X and Y) electromechanical positioning, high speed axial electronic beam steering with lateral electromechanical positioning, high speed beam steering in 3D space, various combinations of including with dynamically varying one or more acoustic cavitation/histotripsy parameters based on the aforementioned ability to update treatment parameters based on threshold measurements (e.g., dynamically adjusting amplitude across the treatment area/volume).

### Other Components, Ancillaries and Accessories

The system may comprise various other components, ancillaries and accessories, including but not limited to computers, computer processors, power supplies including high voltage power supplies, controllers, cables, connectors, networking devices, software applications for security, communication, integration into information systems including hospital information systems, cellular communication devices and modems, handheld wired or wireless controllers, goggles or glasses for advanced visualization, augmented or virtual reality applications, cameras, sensors, tablets, smart devices, phones, internet of things enabling capabilities, specialized use “apps” or user training materials and applications (software or paper based), virtual proctors or trainers and/or other enabling features, devices, systems or applications, and/or methods of using the above.

### System Variations and Methods/Applications

In addition to performing a breadth of procedures, the system may allow additional benefits, such as enhanced planning, imaging and guidance to assist the user. In one embodiment, the system may allow a user to create a patient, target and application specific treatment plan, wherein the system may be configured to optimize treatment parameters based on feedback to the system during planning, and where planning may further comprise the ability to run various test protocols to gather specific inputs to the system and plan.

Feedback may include various energy, power, location, position, tissue and/or other parameters.

The system, and the above feedback, may also be further configured and used to autonomously (and robotically) execute the delivery of the optimized treatment plan and protocol, as visualized under real-time imaging during the procedure, allowing the user to directly observe the local treatment tissue effect, as it progresses through treatment, and start/stop/modify treatment at their discretion. Both test and treatment protocols may be updated over the course of the procedure at the direction of the user, or in some embodiments, based on logic embedded within the system.

It is also recognized that many of these benefits may further improve other forms of acoustic therapy, including thermal ablation with high intensity focused ultrasound (HIFU), high intensity therapeutic ultrasound (HITU) including boiling histotripsy (thermal cavitation), and are considered as part of this disclosure. The disclosure also considers the application of histotripsy as a means to activate previously delivered in active drug payloads whose activity is inert due to protection in a micelle, nanostructure or similar protective structure or through molecular arrangement that allows activation only when struck with acoustic energy.

In another aspect, the Therapy sub-system, comprising in part, one or more amplifiers, transducers and power supplies, may be configured to allow multiple acoustic cavitation and histotripsy driving capabilities, affording specific benefits based on application, method and/or patient specific use. These benefits may include, but are not limited to, the ability to better optimize and control treatment parameters, which may allow delivery of more energy, with more desirable thermal profiles, increased treatment speed and reduced procedure times, enable electronic beam steering and/or other features.

This disclosure also includes novel systems and concepts as related to systems and sub-systems comprising new and “universal” amplifiers, which may allow multiple driving approaches (e.g., single and multi-cycle pulsing). In some embodiments, this may include various novel features to further protect the system and user, in terms of electrical safety or other hazards (e.g., damage to transducer and/or amplifier circuitry).

In another aspect, the system, and Therapy sub-system, may include a plethora of therapy transducers, where said therapy transducers are configured for specific applications and uses and may accommodate treating over a wide range of working parameters (target size, depth, location, etc.) and may comprise a wide range of working specifications (detailed below). Transducers may further adapt, interface and connect to a robotically-enabled system, as well as the Coupling sub-system, allowing the transducer to be positioned within, or along with, an acoustic coupling device allowing, in many embodiments, concurrent imaging and histotripsy treatments through an acceptable acoustic window. The therapy transducer may also comprise an integrated imaging probe or localization sensors, capable of displaying and determining transducer position within the treatment site and affording a direct field of view (or representation of) the treatment site, and as the acoustic cavitation/histotripsy tissue effect and bubble cloud may or may not change in appearance and intensity, throughout the treatment, and as a function of its location within said treatment (e.g., tumor, healthy tissue surrounding, critical structures, adipose tissue, etc.).

The systems, methods and use of the system disclosed herein, may be beneficial to overcoming significant unmet needs in the areas of soft tissue ablation, oncology, immuno-oncology, advanced image guided procedures, surgical procedures including but not limited to open, laparoscopic, single incision, natural orifice, endoscopic, non-invasive, various combination of, various interventional spaces for catheter-based procedures of the vascular, cardiovascular pulmonary and/or neurocranial-related spaces, cosmetics/aesthetics, metabolic (e.g., type 2 diabetes), plastic and reconstructive, ocular and ophthalmology, orthopedic, gynecology and men's health, and other systems, devices and methods of treating diseased, injured, undesired, or healthy tissues, organs or cells.

Systems and methods are also provided for improving treatment patterns within tissue that can reduce treatment time, improve efficacy, and reduce the amount of energy and prefocal tissue heating delivered to patients.

### Use Environments

The disclosed system, methods of use, and use of the system, may be conducted in a plethora of environments and settings, with or without various support systems such as anesthesia, including but not limited to, procedure suites, operating rooms, hybrid rooms, in and out-patient settings, ambulatory settings, imaging centers, radiology, radiation therapy, oncology, surgical and/or any medical center, as well as physician offices, mobile healthcare centers or systems, automobiles and related vehicles (e.g., van), aero and marine transportation vehicles such as planes and ships, and/or any structure capable of providing temporary procedure support (e.g., tent). In some cases, systems and/or sub-systems disclosed herein may also be provided as integrated features into other environments, for example, the direct integration of the histotripsy Therapy sub-system into a MRI scanner or patient surface/bed, wherein at a minimum the therapy generator and transducer are integral to such, and in other cases wherein the histotripsy configuration further includes a robotic positioning system, which also may be integral to a scanner or bed centered design.

### Coupling

Systems may comprise a variety of Coupling sub-system embodiments, of which are enabled and configured to allow acoustic coupling to the patient to afford effective acoustic access for ultrasound visualization and acoustic cavitation/histotripsy (e.g., provide acoustic window and medium between the transducer(s) and patient, and support of). These may include different form factors of such, including open and enclosed device solutions, and some arrangements which may be configured to allow dynamic control over the acoustic medium (e.g., temperature, dissolved gas content, level of particulate filtration, sterility, volume, composition, etc.). Such dynamic control components may be directly integrated to the system (within the Cart), or may be in temporary/intermittent or continuous communication with the system, but externally situated in a separate device and/or cart.

The Coupling sub-system typically comprises, at a minimum, coupling medium (e.g., degassed water or water solutions), a reservoir/container to contain said coupling medium, and a support structure (including interfaces to other surfaces or devices). In most embodiments, the coupling medium is water, and wherein the water may be conditioned before or during the procedure (e.g., chilled, degassed, filtered, etc.). Various conditioning parameters may be employed based on the configuration of the system and its intended use/application.

The reservoir or medium container may be formed and shaped to various sizes and shapes, and to adapt/conform to the patient, allow the therapy transducer to engage/access and work within the acoustic medium, per defined and required working space (minimum volume of medium to allow the therapy transducer to be positioned and/or move through one or more treatment positions or patterns, and at various standoffs or depths from the patient, etc.), and wherein said reservoir or medium container may also mechanically support the load, and distribution of the load, through the use of a mechanical and/or electromechanical support structure. As a representative example, this may include a support frame. The container may be of various shapes, sizes, curvatures, and dimensions, and may be comprised of a variety of materials compositions (single, multiple, composites, etc.), of which may vary throughout. In some embodiments, it may comprise features such as films, drapes, membranes, bellows, etc. that may be insertable and removable, and/or fabricated within, of which may be used to conform to the patient and assist in confining/containing the medium within the container. It may further contain various sensors (e.g., volume/fill level), drains (e.g., inlet/outlet), lighting (e.g., LEDs), markings (e.g., fill lines, set up orientations, etc.), text (e.g., labeling), etc.

In one embodiment, the reservoir or medium container contains a sealable frame, of which a membrane and/or film may be positioned within, to afford a conformable means of contacting the reservoir (later comprising the treatment head/therapy transducer) as an interface to the patient, that further provides a barrier to the medium (e.g., water) between the patient and therapy transducer). In other embodiments, the membrane and/or film may comprise an opening, the patient contacting edge of which affords a fluid/mechanical seal to the patient, but in contrast allows medium communication directly with the patient (e.g., direct degassed water interface with patient). The superstructure of the reservoir or medium container in both these examples may further afford the proximal portion of the structure (e.g., top) to be open or enclosed (e.g., to prevent spillage or afford additional features).

Disclosed membranes may be comprised of various elastomers, viscoelastic polymers, thermoplastics, thermoplastic elastomers, thermoset polymers, silicones, urethanes, rigid/flexible co-polymers, block co-polymers, random block co-polymers, etc. Materials may be hydrophilic, hydrophobic, surface modified, coated, extracted, etc., and may also contain various additives to enhance performance, appearance or stability. In some embodiments, the thermoplastic elastomer may be styrene-ethylene-butylene-styrene (SEBS), or other like strong and flexible elastomers. The membrane form factor can be flat or pre-shaped prior to use. In other embodiments, the membrane could be inelastic (i.e., a convex shape) and pressed against the patient's skin to acoustically couple the transducer to the tissue. Systems and methods are further disclosed to control the level of contaminants (e.g., particulates, etc.) on the membrane to maintain the proper level of ultrasound coupling. Too many particulates or contaminants can cause scattering of the ultrasound waves. This can be achieved with removable films or coatings on the outer surfaces of the membrane to protect against contamination.

Said materials may be formed into useful membranes through molding, casting, spraying, ultrasonic spraying, extruding, and/or any other processing methodology that produces useful embodiments. They may be single use or reposable/reusable. They may be provided non-sterile, aseptically cleaned or sterile, where sterilization may comprise any known method, including but not limited to ethylene oxide, gamma, e-beam, autoclaving, steam, peroxide, plasma, chemical, etc. Membranes can be further configured with an outer molded or over molded frame to provide mechanical stability to the membrane during handling including assembly, set up and take down of the coupling sub-system. Various parameters of the membrane can be optimized for this method of use, including thickness, thickness profile, density, formulation (e.g., polymer molecular weight and copolymer ratios, additives, plasticizers, etc.), including optimizing specifically to maximize acoustic transmission properties, including minimizing impact to cavitation initiation threshold values, and/or ultrasound imaging artifacts, including but not limited to membrane reflections, as representative examples.

Open reservoirs or medium containers may comprise various methods of filling, including using pre-prepared medium or water, that may be delivered into the containers, in some cases to a defined specification of water (level of temperature, gas saturation, etc.), or they may comprise additional features integral to the design that allow filling and draining (e.g., ports, valves, hoses, tubing, fittings, bags, pumps, etc.). These features may be further configured into or to interface to other devices, including for example, a fluidics system. In some cases, the fluidics system may be an in-house medium preparation system in a hospital or care setting room, or conversely, a mobile cart-based system which can prepare and transport medium to and from the cart to the medium container, etc.

Enclosed iterations of the reservoir or medium container may comprise various features for sealing, in some embodiments sealing to a proximal/top portion or structure of a reservoir/container, or in other cases where sealing may comprise embodiments that seal to the transducer, or a feature on the transducer housings. Further, some embodiments may comprise the dynamic ability to control the volume of fluid within these designs, to minimize the potential for air bubbles or turbulence in said fluid and to allow for changes in the focal length to the target area without moving the transducer. As such, integrated features allowing fluid communication, and control of, may be provided (ability to provide/remove fluid on demand), including the ability to monitor and control various fluid parameters, some disclosed above. In order to provide this functionality, the overall system, and as part, the Coupling sub-system, may comprise a fluid conditioning system, which may contain various electromechanical devices, systems, power, sensing, computing, pumping, filtering and control systems, etc. The reservoir may also be configured to receive signals that cause it to deform or change shape in a specific and controlled manner to allow the target point to be adjusted without moving the transducer.

Coupling support systems may include various mechanical support devices to interface the reservoir/container and medium to the patient, and the workspace (e.g., bed, floor, etc.). In some embodiments, the support system comprises a mechanical arm with 3 or more degrees of freedom. Said arm may have a proximal interface with one or more locations (and features) of the bed, including but not limited to, the frame, rails, customized rails or inserts, as well as one or more distal locations of the reservoir or container. The arm may also be a feature implemented on one or more Carts, wherein Carts may be configured in various unlimited permutations, in some cases where a Cart only comprises the role of supporting and providing the disclosed support structure.

In some embodiments, the support structure and arm may be a robotically-enabled arm, implemented as a stand-alone Cart, or integrated into a Cart further comprising two or more system sub-systems, or where in the robotically-enabled arm is an arm of another robot, of interventional, surgical or other type, and may further comprise various user input features to actuate/control the robotic arm (e.g., positioning into/within coupling medium) and/or Coupling solution features (e.g., filling, draining, etc.). In some examples, the support structure robotic arm positional encoders may be used to coordinate the manipulation of the second arm (e.g. comprising the therapy transducer/treatment head), such as to position the therapy transducer to a desired/known location and pose within the coupling support structure.

Overall, significant unmet needs exist in interventional and surgical medical procedures today, including those procedures utilizing minimally invasive devices and approaches to treat disease and/or injury, and across various types of procedures where the unmet needs may be solved with entirely new medical procedures. Today's medical system capabilities are often limited by access, wherein a less or non-invasive approach would be preferred, or wherein today's tools aren't capable to deliver preferred/required tissue effects (e.g., operate around/through critical structures without serious injury), or where the physical set up of the systems makes certain procedure approaches less desirable or not possible, and where a combination of approaches, along with enhanced tissue effecting treatments, may enable entirely new procedures and approaches, not possible today.

In addition, specific needs exist for enabling histotripsy delivery, including robotic histotripsy delivery, wherein one or more histotripsy therapy transducers may be configured to acoustically couple to a patient, using a completely sealed approach (e.g., no acoustic medium communication with the patient's skin) and allowing the one or more histotripsy transducers to be moved within the coupling solution without impeding the motion/movement of the robotic arm or interfering/disturbing the coupling interface, which could affect the intended treatment and/or target location.

Disclosed herein are histotripsy acoustic and patient coupling systems and methods, to enable histotripsy therapy/treatment, as envisioned in any setting, from interventional suite, operating room, hybrid suites, imaging centers, medical centers, office settings, mobile treatment centers, and/or others, as non-limiting examples. The following disclosure further describes novel systems used to create, control, maintain, modify/enhance, monitor and setup/takedown acoustic and patient coupling systems, in a variety of approaches, methods, environments, architectures and work-flows. In general, the disclosed novel systems may allow for a coupling medium, in some examples degassed water, to be interfaced between a histotripsy therapy transducer and a patient, wherein the acoustic medium provides sufficient acoustic coupling to said patient, allowing the delivery of histotripsy pulses through a user desired treatment location (and volume), where the delivery may require physically moving the histotripsy therapy transducer within a defined work-space comprising the coupling medium, and also where the coupling system is configured to allow said movement of the therapy transducer (and positioning system, e.g., robot) freely and unencumbered from by the coupling support system (e.g., a frame or manifold holding the coupling medium).

### Coupling System and Sub-Systems/Components

The disclosed histotripsy acoustic and patient coupling systems, in general, may comprise one or more of the following sub-systems and components, including but not limited to 1) a membrane/barrier film to provide an enclosed, sealed and conformal patient coupling and histotripsy system interface, 2) a frame and assembly to retain the membrane and provide sufficient work and head space for a histotripsy therapy transducers required range of motion (x, y and z, pitch, roll and yaw), 3) a sufficient volume of ultrasound medium to afford acoustic coupling and interfaces to a histotripsy therapy transducer and robotic arm, 4) one or more mechanical support arms to allow placement, positioning and load support of the frame, assembly and medium and 5) a fluidics system to prepare, provide and remove ultrasound medium(s) from the frame and assembly.

In some embodiments, the coupling system may be fully sealed, and in other embodiments and configurations, it may be partially open to afford immediate access (physical and/or visual).

The acoustic and patient coupling systems and sub-systems may further comprise various features and functionality, and associated work-flows, and may also be configured in a variety of ways to enable histotripsy procedures as detailed below.

The therapy and/or imaging transducers can be housed in a coupling assembly which can further include a coupling membrane and a membrane constraint configured to prevent the membrane from expanding too far from the transducer. The coupling membrane can be filled with an acoustic coupling medium such as a fluid or a gel. The membrane constraint can be, for example, a semi-rigid or rigid material configured to restrict expansion/movement of the membrane. In some embodiments, the membrane constraint is not used, and the elasticity and tensile strength of the membrane prevent over expansion. The coupling membrane can be a mineral-oil infused SEBS membrane to prevent direct fluid contact with the patient's skin. In the illustrated embodiment, the coupling assembly is supported by a mechanical support arm which can be load bearing in the x-y plane but allow for manual or automated z-axis adjustment. The mechanical support arm can be attached to the floor, the patient table, or the cart. The mechanical support is designed and configured to conform and hold the coupling membrane in place against the patient's skin while still allowing movement of the therapy/imaging transducer relative to the patient and also relative to the coupling membrane with the robotic positioning arm.

The system can further include a fluidics system that can include a fluid source, a cooling and degassing system, and a programmable control system. The fluidics system is configured for external loading of the coupling membrane with automated control of fluidic sequences. Further details on the fluidics system are provided below.

### Membranes/Barrier Films and Related Architectures

Membranes and barrier films may be composed of various biocompatible materials which allow conformal coupling to patient anatomy with minimal or no entrapped bubbles capable of interfering with ultrasound imaging and histotripsy therapy, and that are capable of providing a sealed barrier layer between said patient anatomy and the ultrasound medium, of which is contained within the work-space provided by the frame and assembly.

Membrane and barrier film materials may comprise flexible and elastomeric biocompatible materials/polymers, such as various thermoplastic and thermoset materials, as well as permanent or bioresorbable polymers. Additionally, the frame of the UMC can also comprise the same materials. In some examples, the membrane may be rigid or semi-rigid polymers which are pre-shaped or flat.

### Ultrasound Medium

As previously described, the ultrasound medium may comprise any applicable medium capable of providing sufficient and useful acoustic coupling to allow histotripsy treatments and enable sufficient clinical imaging (e.g., ultrasound). Ultrasound mediums, as a part of this disclosure and system, may comprise, but are not limited to, various aqueous solutions/mediums, including mixtures with other co-soluble fluids, of which may have preferred or more preferred acoustic qualities, including ability to match speed of sound, etc. Example mediums may comprise degassed water and/or mixtures/co-solutions of degassed water and various alcohols, such as ethanol.

### Mechanical Support Arms and Arm Architectures

In order to support the acoustic and patient coupling system, including providing efficient and ergonomic work-flows for users, various designs and configurations of mechanical support arms (and arm architectures) may be employed. Support arms may be configured with a range of degrees of freedom, including but not limited to allowing, x, y, z, pitch, roll and yaw, as well additional interfacing features that may allow additional height adjustment or translation.

Arms may comprise a varied number and type of joints and segments. Typically, arms may comprise a minimum of 2 segments. In some configurations, arms may comprise 3 to 5 segments.

Arms are also be configured to interface proximally to a main support base or base interface (e.g., robot, table, table/bed rail, cart, floor mount, etc.) and distally to the frame/assembly and overall “UMC” or “coupling solution”. This specific distal interface may further include features for controlling position/orientation of the frame/assembly, at the frame/assembly interface.

For example, in some embodiments, the arm/frame interface may comprise a ball joint wrist. In another example, the interface may include use of a gimbal wrist or an adjustable pitch and roll controlled wrist. These interfaces may be further employed with specific user interfaces and inputs, to assist with interacting with the various wrists, of which may include additional handles or knobs (as an unlimited example), to further enable positioning the UMC/coupling solution. For example, a gimbal wrist may benefit from allowing the frame/assembly to have 3 degrees of freedom (independent of the arm degrees of freedom), including pitch, roll and yaw adjustments.

Support arms, configured with arm wrists, further interfaced with frames/assemblies, may comprise features such as brakes, including cable or electronic actuated brakes, and quick releases, which may interact with one or more axis, individually, or in groupings. They may also include electronic lift systems and base supports. In some embodiments, these lift systems/base supports are co-located with robot arm bases, wherein said robot arm is equipped with the histotripsy therapy transducer configured to fit/work within the enclosed coupling solution. In other embodiments, the support arm is located on a separate cart. In some cases, the separate cart may comprise a fluidics system or user console. In other embodiments, it is interfaced to a bed/table, including but not limited to a rail, side surface, and/or bed/table base. In other examples/embodiments, it's interfaced to a floor-based structure/footing, capable of managing weight and tipping requirements.

### Fluidics Systems, Control Systems and System Architectures

As a part of overall fluidics management, histotripsy systems including acoustic/patient coupling systems, may be configured to include an automated fluidics system, which primarily is responsible for providing a reservoir for preparation and use of coupling medium, where preparation may include the ability to degas, chill, monitor, adjust, dispense/fill, and retrieve/drain coupling medium to/from the frame/assembly. The fluidics system may include an emergency high flow rate system for rapid draining of the coupling medium from the UMC. In some embodiments, the fluidics system can be configured for a single use of the coupling medium, or alternatively, for re-use of the medium. In some embodiments, the fluidics system can implement positive air pressure or vacuum to carry out leak tests of the UMC and membrane prior to filling with a coupling medium. Vacuum assist can also be used for removal of air from the UMC during the filling process. The fluidics system can further include filters configured to prevent particulate contamination from reaching the UMC.

The fluidics system may implemented in the form of a mobile fluidics cart. The cart may comprise an input tank, drain tank, degassing module, fill pump, drain pump, inert gas tank, air compressor, tubing/connectors/lines, electronic and manual controls systems and input devices, power supplies and one or more batteries. The cart in some cases may also comprise a system check vessel/reservoir for evaluating histotripsy system performance and related system diagnostics (configured to accommodate a required water volume and work-space for a therapy transducer).

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

1. An ultrasound device, comprising:
a flat transducer array comprising one or more transducer elements;
a dual-material acoustic lens coupled to the flat transducer array, the dual-material acoustic lens having a flat coupling surface and comprising a first material having a first speed of sound therein and a second material having a second speed of sound therein, the dual-material acoustic lens being configured to provide an equal time of flight between the flat transducer array and a focus of the ultrasound device for any radial distance across the flat transducer array.

2. The ultrasound device of claim 1, wherein the dual-material acoustic lens has a constant overall thickness.

3. The ultrasound device of claim 1, wherein the first speed of sound in the first material is higher than the second speed of sound in the second material.

4. The ultrasound device of claim 1, wherein the first speed of sound is greater than 1500 m/s.

5. The ultrasound device of claim 1, wherein the second speed of sound is less than 1500 m/s.

6. The ultrasound device of claim 1, wherein the first material has a concave shape.

7. The ultrasound device of claim 1, wherein the first material has an elliptical shape.

8. The ultrasound device of claim 1, wherein the first material comprises 3D printed plastic.

9. The ultrasound device of claim 8, wherein the second material comprises a silicon fill material.

10. The ultrasound device of claim 1, wherein the dual-material acoustic lens comprises a Fresnel lens.

11. The ultrasound device of claim 10, wherein the first material is shaped to include a plurality of steps.

12. The ultrasound device of claim 11, wherein each of the plurality of steps has a thickness that is an integer multiple of a wavelength of the flat transducer array.

13. The ultrasound device of claim 1, wherein the dual-material acoustic lens has a thickness of less than 15 mm.

14. The ultrasound device of claim 1, wherein the dual-material acoustic lens has a thickness of less than 13 mm.

15. The ultrasound device of claim 1, wherein the dual-material acoustic lens has a thickness of less than 5 mm.

16. The ultrasound device of claim 1, wherein the dual-material acoustic lens has a thickness of less than 4 mm.

17. The ultrasound device of claim 1, wherein the dual-material acoustic lens and the flat transducer array are disposed on or in a probe housing.

18. The ultrasound device of claim 1, wherein the dual-material acoustic lens provides for dry coupling of the flat transducer array to a subject.

19. The ultrasound device of claim 18, wherein the device is configured to deliver histotripsy pulses into the subject's tissue to generate cavitation at the focus.

20. The ultrasound device of claim 1, further comprising an ultrasound imaging transducer array integrated into a central location of the dual-material acoustic lens and/or the flat transducer array.

21.-50. (canceled)
